(19) United States
(12) Patent Application Publication
(10) Pub. No.: US 2022/0227700 A1
(43) Pub. Date: Jul. 21, 2022

(54) HDAC3 CATALYTIC INHIBITOR DEVELOPMENT AND USES THEREOF

(71) Applicant: DANA-FARBER CANCER INSTITUTE, INC., Boston, MA (US)

(72) Inventors: Deyao Li, Boston, MA (US); Paul Park, Waltham, MA (US); Jun Qi, Sharon, MA (US); Lei Wu, Boston, MA (US)

(21) Appl. No.: 17/596,466

(22) PCT Filed: Jun. 12, 2020
(86) PCT No.: PCT/US20/37510

§ 371 Date: Dec. 10, 2021

Related U.S. Application Data
(60) Provisional application No. 62/860,927, filed on Jun. 13, 2019.

(51) Int. Cl. C07C 233/81; C07D 495/04; C07D 213/40; C07D 215/38; C07D 209/86; C07D 277/82; C07D 217/02; C07D 333/66; C07C 317/32; C07D 211/58; C07D 233/61; A61P 35/00

(52) U.S. Cl. CPC C07C 233/81; C07D 495/04; C07D 213/40; C07D 215/38; C07D 209/86; A61P 35/00; C07D 217/02; C07D 333/66; C07C 317/32; C07D 211/58; C07D 233/61; C07D 277/82

(57) ABSTRACT

Provided herein are compounds, pharmaceutical compositions comprising such compounds, and methods of using such compounds to treat diseases or disorders associated with HDAC3 activity.

### RELATED APPLICATIONS

This application claims priority to U.S. Provisional Application No. 62/860,927 filed of Jun. 13, 2019, the entire content of which is incorporated by reference.

### BACKGROUND

Identity determining transcription factors (TFs), or core regulatory (CR) TFs, are governed by cell-type specific super enhancers (SEs). The characterization of drug-like small molecules to selectively inhibit core regulatory circuitry is of high interest for treatment of cancers. In alveolar rhabdomyosarcoma, PAX3-FOXO1 activates SEs to induce the expression of other core CR TFs, providing a model system for studying cancer cell addiction to CR transcription. Chemical probes along the acetylation-axis are able to cause selective disruption of CR transcription. Histone deacetylase (HDAC) enzymes, which remove acetylation are the most selective for CR TF transcription. Eleven human HDACs, which use Zn as a cofactor, have been identified (Taunton et al. Science 1996, 272, 408-411; Yang et al. J. Biol. Chem. 1997, 272, 28001-28007. Grozinger et al. Proc. Natl. Acad. Sci. U.S.A. 1999, 96, 4868-4873; Kao et al. Genes Dev. 2000, 14, 55-66. Hu et al. J. Biol. Chem. 2000, 275, 15254-15264; Zhou et al. Proc. Nat. Acad. Sci U.S.A. 2001, 98, 10572-10577; Venter et al. Science 2001, 291, 1304-1351) and these members fall into three classes (class I, II, and IV) based on sequence homology to their yeast orthologues (O. Witt et al. Cancer Letters, 2009, 277, 8-21). Class I HDACs include HDAC1, HDAC2, HDAC3, and HDAC8, and are referred to as “classical” HDACs, which implies a catalytic pocket with a Zn²⁺ ion at its base. HDAC1/2/3 are the isoforms that halt CR transcription by making CR TF sites hyper-accessible and disrupting chromatin looping. This counterintuitive regulation occurs due to the unique transcriptional apparatus requirements at CR TF genes. The CR requirements found herein are likely generalizable to other cancers, and provides a new mechanistic framework for interpreting chemical epigenomics.

There remains a need for preparing structurally diverse HDAC inhibitors, particularly ones that are potent and/or selective inhibitors of particular classes of HDACs and individual HDACs.

### SUMMARY

Provided herein are compounds and methods of using these compounds to treat disorders related to HDAC3 function, including cancer and neurodegenerative diseases.

In an aspect, provided herein are compounds of Formula I:

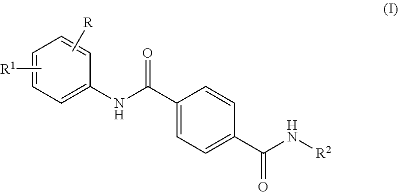

or a pharmaceutically acceptable salt thereof;

wherein:

R is selected from the group consisting of fluoro, bromo, chloro, —NH₂, —OH, —SH, —NHR³, —N(R³)₂, OR³, SR³, NO₂, thienyl, and CN;

R¹ is selected from the group consisting of fluoro, bromo, chloro, —NH₂, —OH, —SH, —NHR³. —N(R³)₂, OR³, SR³, NO₂, thienyl, and CN;

R² is selected from the group consisting of C₆-C₁₀ aryl, C₅-C₁₃ heteroaryl, C₃-C₁₀ cycloalkyl, C₃-C₁₀ heterocycloalkyl, C₁-C₆ alkyl-C₆-C₁₀ aryl, C₁-C₆ alkyl-C₅-C₁₃ heteroaryl, C₁-C₆ alkyl-C₃-C₁₀ cycloalkyl, C₁-C₆ alkyl-C₃-C₁₀ heterocycloalkyl, and -linker-biotin;

wherein C₅-C₁₃ heteroaryl, C₆-C₁₀ aryl, and C₁-C₆ alkyl are optionally substituted with one to three halo, phenyl, —C(O)Me, —OMe, methyl, NO₂, —SO₂Me, Ce heterocycloalkyl, C₅-C₆ heteroaryl, and CF₃; and

R³ is independently, at each occurrence, selected from the group consisting of H, C₁-C₆ alkyl, and C₁-C₆ alkoxy.

In an embodiment, R is selected from the group consisting of fluoro, bromo, chloro, —NH₂, —OH, and —SH; R¹ is selected from the group consisting of fluoro, bromo, chloro, —NH₂, —OH, and —SH; and R² is C₅-C₁₀ aryl or C₅-C₁₃ heteroaryl. In another embodiment, R is fluoro and R¹ is —NH₂. In yet another embodiment, R² is C₅-C₁₃ heteroaryl.

In still another embodiment, provided herein are compounds Formula I that are compounds of Formula II:

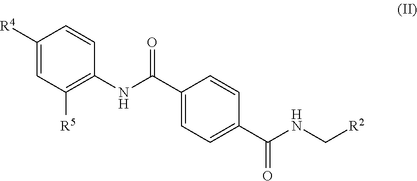

or a pharmaceutically acceptable salt thereof;

wherein:

R² is selected from the group consisting of C₆-C₁₀ aryl, C₅-C₁₀ heteroaryl, or linker-biotin, wherein C₆-C₁₀ aryl is optionally substituted with halo or SO₂Me;

R⁴ is selected from the group consisting of fluoro, bromo, chloro, —NH₂, —OH, and —SH; and

R⁵ is selected from the group consisting of fluoro, bromo, chloro, —NH₂, —OH, and —SH.

In an embodiment of Formula II, the linker has the following formula:

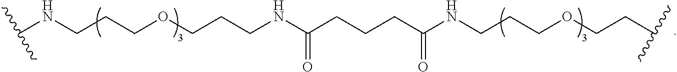

In an embodiment of Formula II, R² is C₅-C₁₀ heteroaryl, R⁴ is fluoro, and R⁵ is —NH₂. In another embodiment, R₂ is

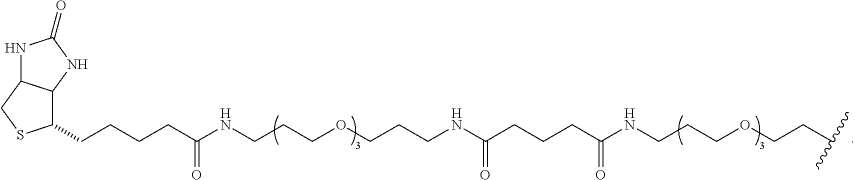

In an embodiment, the compound of Formula I or Formula II is a compound of Formula III:

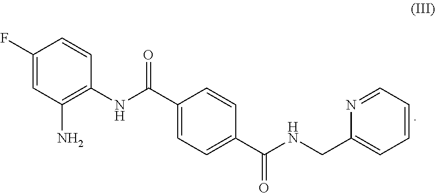 [SMILES: Nc1cc(F)ccc1NC(=O)c1ccc(C(=O)NCc2ccccn2)cc1]

or a pharmaceutically acceptable salt thereof.

In another embodiment, the compound of Formula I is a compound of Formula IV:

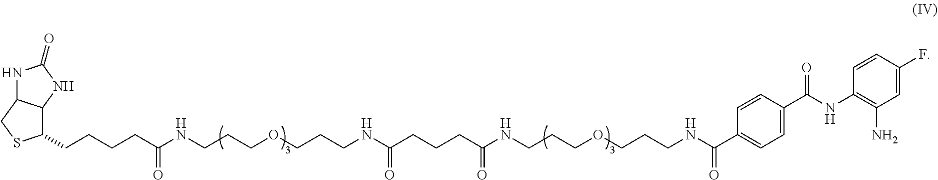

In yet another aspect, provided herein are pharmaceutical compositions comprising any of the compounds described herein, or a pharmaceutically acceptable salt thereof, together with a pharmaceutically acceptable carrier.

In an embodiment, a compound of Formula I is selected from the group consisting of:

TABLE 1

| Structure | Compound Name |
|---|---|
| 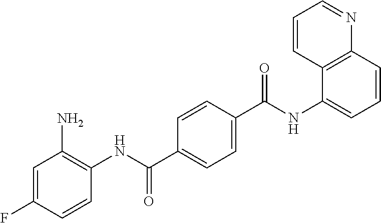 [SMILES: Nc1cc(F)ccc1NC(=O)c1ccc(C(=O)Nc2cccc3ncccc23)cc1] | DL-HDAC1 |
| 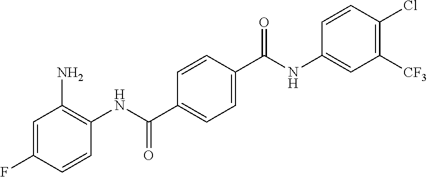 [SMILES: Nc1cc(F)ccc1NC(=O)c1ccc(C(=O)Nc2ccc(Cl)c(C(F)(F)F)c2)cc1] | DL-HDAC2 |
| 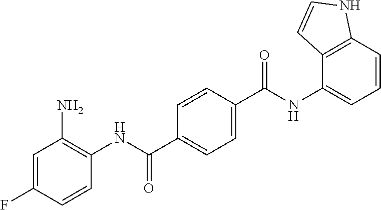 [SMILES: Nc1cc(F)ccc1NC(=O)c1ccc(C(=O)Nc2cccc3[nH]ccc23)cc1] | DL-HDAC3 |
| 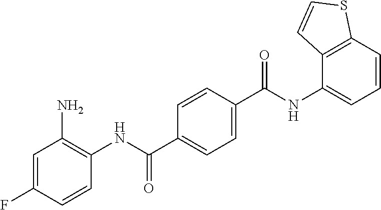 [SMILES: Nc1cc(F)ccc1NC(=O)c1ccc(C(=O)Nc2cccc3sccc23)cc1] | DL-HDAC4 |
| 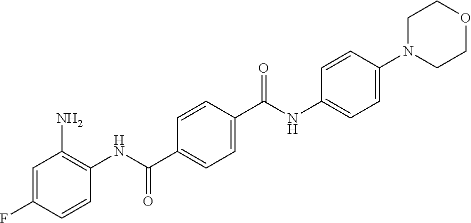 [SMILES: Nc1cc(F)ccc1NC(=O)c1ccc(C(=O)Nc2ccc(N3CCOCC3)cc2)cc1] | DL-HDAC5 |
| 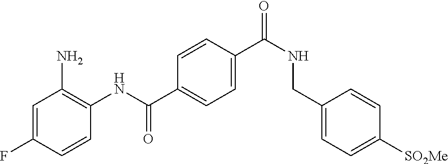 [SMILES: CS(=O)(=O)c1ccc(CNC(=O)c2ccc(C(=O)Nc3ccc(F)cc3N)cc2)cc1] | DL-HDAC6 |
| 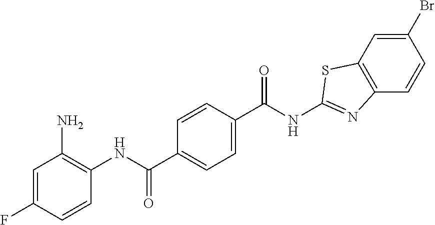 [SMILES: Nc1cc(F)ccc1NC(=O)c1ccc(C(=O)Nc2nc3ccc(Br)cc3s2)cc1] | DL-HDAC7 |
| 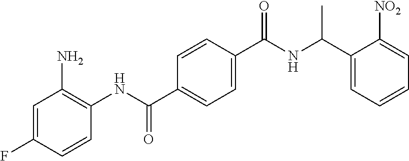 [SMILES: CC(NC(=O)c1ccc(C(=O)Nc2ccc(F)cc2N)cc1)c1ccccc1[N+](=O)[O-]] | DL-HDAC8 |
| 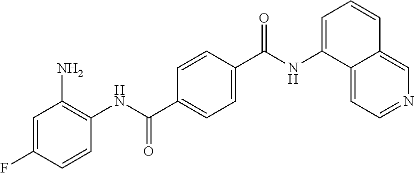 [SMILES: Nc1cc(F)ccc1NC(=O)c1ccc(C(=O)Nc2cccc3cnccc23)cc1] | DL-HDAC9 |
| 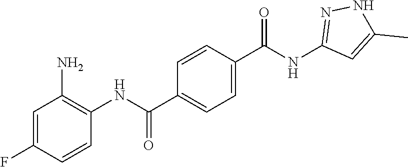 [SMILES: Cc1cc(NC(=O)c2ccc(C(=O)Nc3ccc(F)cc3N)cc2)n[nH]1] | DL-HDAC10 |
| 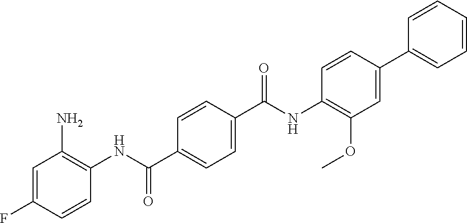 [SMILES: COc1cc(-c2ccccc2)ccc1NC(=O)c1ccc(C(=O)Nc2ccc(F)cc2N)cc1] | DL-HDAC11 |
| 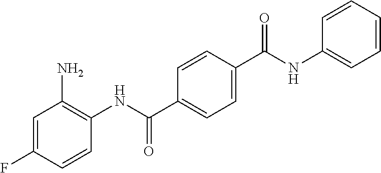 [SMILES: Nc1cc(F)ccc1NC(=O)c1ccc(C(=O)Nc2ccccc2)cc1] | DL-HDAC12 |
| 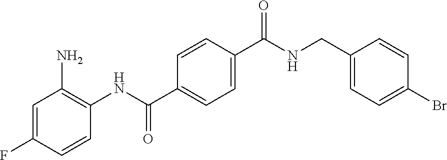 [SMILES: Nc1cc(F)ccc1NC(=O)c1ccc(C(=O)NCc2ccc(Br)cc2)cc1] | DL-HDAC13 |
| 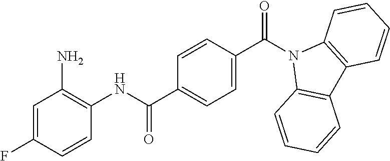 [SMILES: Nc1cc(F)ccc1NC(=O)c1ccc(C(=O)n2c3ccccc3c3ccccc32)cc1] | DL-HDAC14 |
| 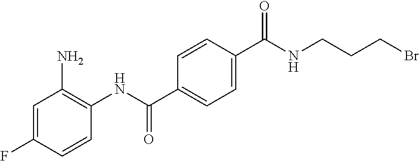 [SMILES: Nc1cc(F)ccc1NC(=O)c1ccc(C(=O)NCCCBr)cc1] | DL-HDAC15 |
| 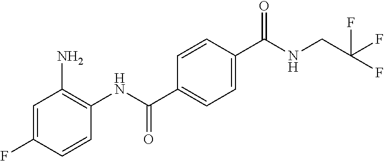 [SMILES: Nc1cc(F)ccc1NC(=O)c1ccc(C(=O)NCC(F)(F)F)cc1] | DL-HDAC16 |
| 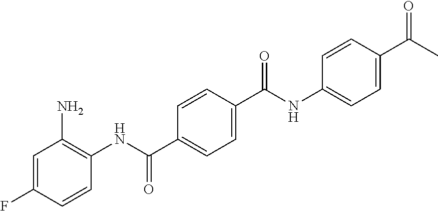 [SMILES: CC(=O)c1ccc(NC(=O)c2ccc(C(=O)Nc3ccc(F)cc3N)cc2)cc1] | DL-HDAC17 |
| 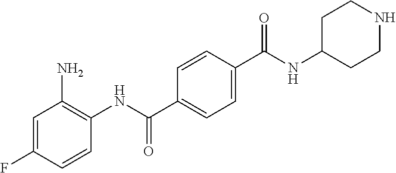 [SMILES: Nc1cc(F)ccc1NC(=O)c1ccc(C(=O)NC2CCNCC2)cc1] | DL-HDAC18 |
| 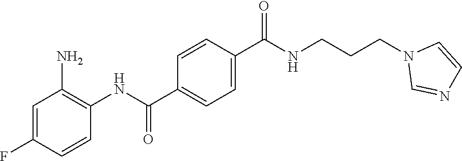 [SMILES: Nc1cc(F)ccc1NC(=O)c1ccc(C(=O)NCCCn2ccnc2)cc1] | DL-HDAC19 |
| 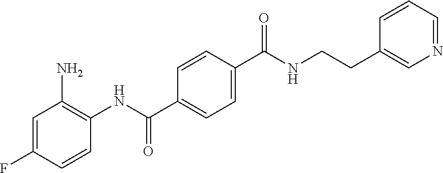 [SMILES: Nc1cc(F)ccc1NC(=O)c1ccc(C(=O)NCCc2cccnc2)cc1] | DL-HDAC20 |
| 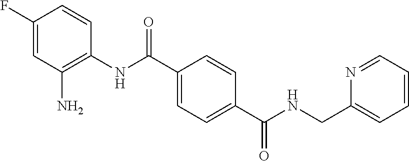 [SMILES: Nc1cc(F)ccc1NC(=O)c1ccc(C(=O)NCc2ccccn2)cc1] | LW3 |
| 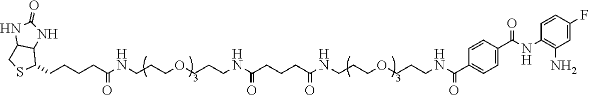 | Biotin- LW3 |

or a pharmaceutically acceptable salt thereof.

In still another aspect, provided herein are methods of inhibiting the activity of histone deacetylase 3 (HDAC3) in an individual in need thereof, comprising administering to the individual any of the compounds or compositions described herein.

In an aspect, provided herein are methods of treating a disease mediated by HDAC3 in an individual in need thereof, comprising administering to the individual any of the compounds or compositions described herein.

In another aspect, provided herein are methods of treating cancer in an individual in need thereof, comprising administering to the individual any of the compounds or compositions described herein.

In an embodiment, the cancer is medulloblastoma, rhabdomyosarcoma, Hodgkin lymphoma, acute myeloid leukemia, myelodysplastic syndrome, pancreatic cancer, colon cancer, ovarian cancer, lung cancer, stomach cancer, a muscle cancer, a bone cancer, or a skin cancer. In another embodiment, the cancer is rhabdomyosarcoma. In yet another embodiment, the cancer is alveloar rhabdomyosarcoma. In still another embodiment, the cancer is pediatric rhabdomyosarcoma.

In an embodiment, the subject is human.

In yet another aspect, provided herein are methods of treating a neurodegenerative disease in an individual in need thereof, comprising administering to the individual a compound of Formula I or a pharmaceutically acceptable salt thereof.

In an embodiment, the neurodegenerative disease is spinal muscular atrophy, polyglutamine-related diseases, or amyotrophic lateral sclerosis. In another embodiment, polyglutamine-related disease is Huntington disease, dentatorubral-pallidoluysian atrophy, or spinocerebellar ataxia type 6 (SCA6).

In still another aspect, provided herein are processes for preparing a compound of Formula V:

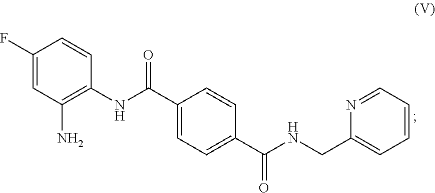 [SMILES: Nc1cc(F)ccc1NC(=O)c1ccc(C(=O)NCc2ccccn2)cc1]

comprising reacting a compound of Formula VI:

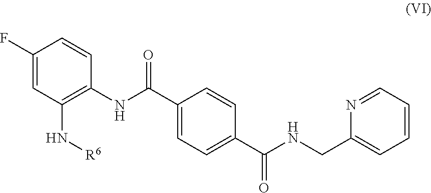

with an acid in a solvent;

wherein

R⁴ is selected from the group consisting of fluoro, bromo, and chloro; and

R⁶ is a protecting group selected from the group consisting of acetyl (Ac), benzyl (Bn), teRt-butyloxycarbonyl (Boc), benzoyl (Bz), carboxybenzyl (Cbz), carbamate, 3,4-dimethoxy-benzyl (DMPM), 9-fluorenylmethyloxycarbonyl (Fmoc), p-methoxybenzyl carbonyl (Moz), 4-nitrobenzylsulfonyl (Nos), p-methoxybenzyl (PMB), p-methoxyphenyl (PMP), 4-toluenesulfonyl (Tos), and trichloroethyl chloroformate (Troc).

In an aspect, provided herein are processes for preparing a compound of Formula VI:

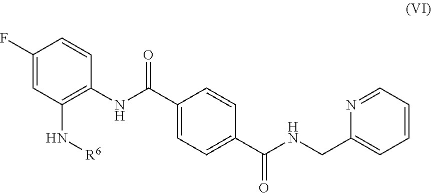

comprising reacting a compound of Formula VII:

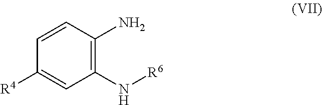

with a compound of Formula VIII:

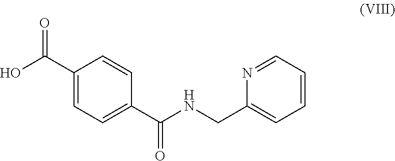 [SMILES: O=C(O)c1ccc(C(=O)NCc2ccccn2)cc1]

in the presence of a peptide coupling reagent, a base, and a solvent;

wherein

R⁴ is selected from the group consisting of fluoro, bromo, and chloro; and

R⁶ is a protecting group selected from the group consisting of acetyl (Ac), benzyl (Bn), tert-butyloxycarbonyl (Boc), benzoyl (Bz), carboxybenzyl (Cbz), carbamate, 3,4-dimethoxy-benzyl (DMPM), 9-fluorenylmethyloxycarbonyl (Fmoc), p-methoxybenzyl carbonyl (Moz), 4-nitrobenzylsulfonyl (Nos), p-methoxybenzyl (PMB), p-methoxyphenyl (PMP), 4-toluenesulfonyl (Tos), and trichloroethyl chloroformate (Troc).

In another aspect, provided herein are processes for preparing a compound of Formula VII:

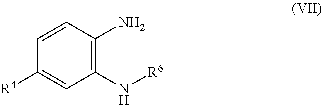

comprising treating a compound of Formula IX:

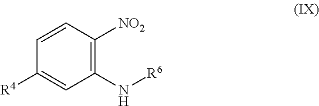

with hydrogen gas in the presence of a palladium catalyst and a solvent or mixture of solvents;

wherein

R⁴ is selected from the group consisting of fluoro, bromo, and chloro; and

R⁶ is a protecting group selected from the group consisting of acetyl (Ac), benzyl (Bn), tert-butyloxycarbonyl (Boc), benzoyl (Bz), carboxybenzyl (Cbz), carbamate, 3,4-dimethoxy-benzyl (DMPM), 9-fluorenylmethyloxycarbonyl (Fmoc), p-methoxybenzyl carbonyl (Moz), 4-nitrobenzylsulfonyl (Nos), p-methoxybenzyl (PMB), p-methoxyphenyl (PMP), 4-toluenesulfonyl (Tos), and trichloroethyl chloroformate (Troc).

In yet another aspect, provided herein are processes for preparing a compound of Formula IX:

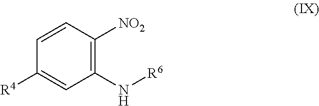

comprising reacting a compound of Formula X:

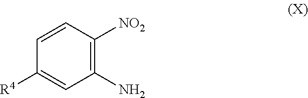

with a protecting group reagent and a base, or combination of bases, in a solvent:

wherein

R⁴ is selected from the group consisting of fluoro, bromo, and chloro; and

R⁶ is a protecting group selected from the group consisting of acetyl (Ac), benzyl (Bn), tert-butyloxycarbonyl (Boc), benzoyl (Bz), carboxybenzyl (Cbz), carbamate, 3,4-dimethoxy-benzyl (DMPM), 9-fluorenylmethyloxycarbonyl (Fmoc), p-methoxybenzyl carbonyl (Moz), 4-nitrobenzylsulfonyl (Nos), p-methoxybenzyl (PMB), p-methoxyphenyl (PMP), 4-toluenesulfonyl (Tos), and trichloroethyl chloroformate (Troc).

In still another aspect, provided herein are processes for preparing a compound of Formula Vii:

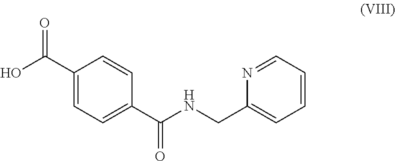 [SMILES: O=C(O)c1ccc(C(=O)NCc2ccccn2)cc1]

comprising reacting a compound of Formula XI:

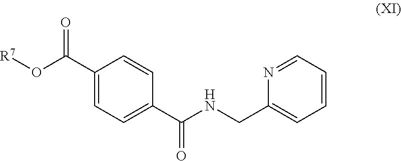

with a base in a solvent,

wherein R⁷ is C₁-C₆ alkyl.

In an aspect, provided herein are processes for preparing a compound of Formula XI:

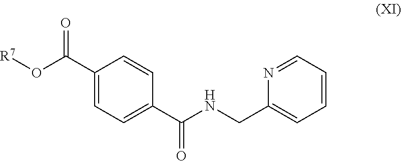

comprising reacting a compound of Formula XII:

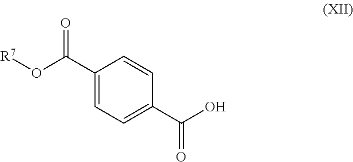

with a compound of Formula XIII:

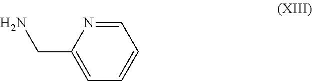

in the presence of a peptide coupling reagent, a base, and a solvent;

wherein R⁷ is C₁-C₆ alkyl.

In another aspect, provided herein are processes for preparing a compound of Formula IV:

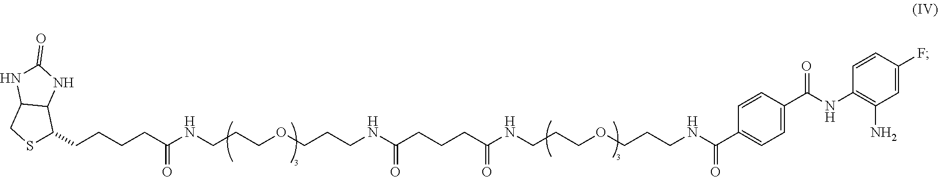

comprising reacting a compound of Formula XIV:

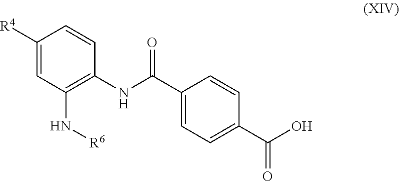

with a compound of Formula XV:

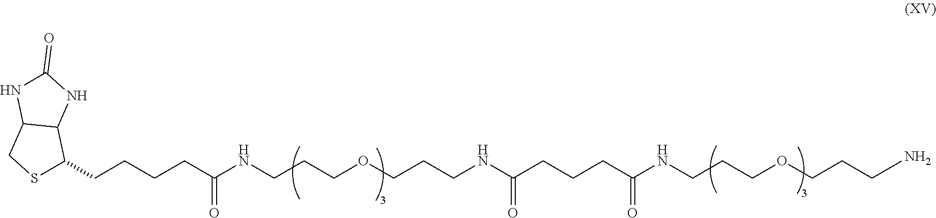

in the presence of a peptide coupling reagent and a base in a solvent, then further reacting the product of the above reaction with an acid;

wherein

R⁴ is selected from the group consisting of fluoro, bromo, and chloro; and

R⁶ is a protecting group selected from the group consisting of acetyl (Ac), benzyl (Bn), tert-butyloxycarbonyl (Boc), benzoyl (Bz), carboxybenzyl (Cbz), carbamate, 3,4-dimethoxy-benzyl (DMPM), 9-fluorenylmethyloxycarbonyl (Fmoc), p-methoxybenzyl carbonyl (Moz), 4-nitrobenzylsulfonyl (Nos), p-methoxybenzyl (PMB), p-methoxyphenyl (PMP), 4-toluenesulfonyl (Tos), and trichloroethyl chloroformate (Troc).

In yet another aspect, provided herein are processes for preparing a compound of Formula XIV:

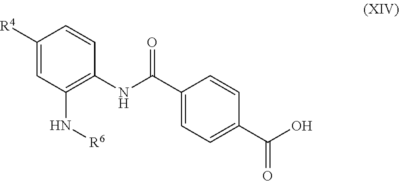

comprising reacting a compound of Formula XVI:

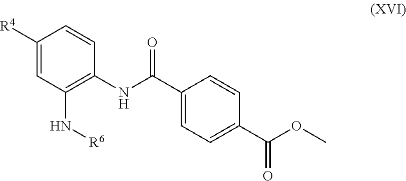

with a base in a solvent;

wherein

R⁴ is selected from the group consisting of fluoro, bromo, and chloro; and

R⁶ is a protecting group selected from the group consisting of acetyl (Ac), benzyl (Bn), tert-butyloxycarbonyl (Boc), benzoyl (Bz), carboxybenzyl (Cbz), carbamate, 3,4-dimethoxy-benzyl (DMPM), 9-fluorenylmethyloxycarbonyl (Fmoc), p-methoxybenzyl carbonyl (Moz), 4-nitrobenzylsulfonyl (Nos), p-methoxybenzyl (PMB), p-methoxyphenyl (PMP), 4-toluenesulfonyl (Tos), and trichloroethyl chloroformate (Troc).

In still another aspect, provided herein are processes for preparing a compound of Formula XVI:

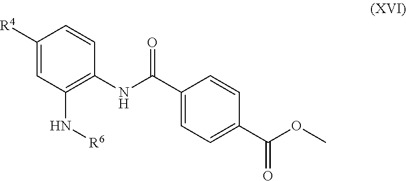

comprising reacting a compound of Formula XVII:

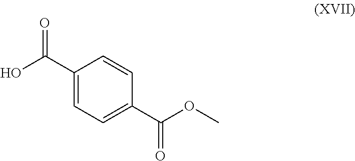

with a compound of Formula VII:

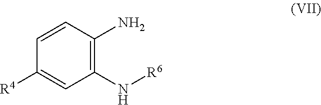

in the presence of a peptide coupling reagent and a base in a solvent;

wherein

R⁴ is selected from the group consisting of fluoro, bromo, and chloro; and

R⁶ is a protecting group selected from the group consisting of acetyl (Ac), benzyl (Bn), tert-butyloxycarbonyl (Boc), benzoyl (Bz), carboxybenzyl (Cbz), carbamate, 3,4-dimethoxy-benzyl (DMPM), 9-fluorenylmethyloxycarbonyl (Fmoc), p-methoxybenzyl carbonyl (Moz), 4-nitrobenzylsulfonyl (Nos), p-methoxybenzyl (PMB), p-methoxyphenyl (PMP), 4-toluenesulfonyl (Tos), and trichloroethyl chloroformate (Troc).

### BRIEF DESCRIPTION OF THE DRAWINGS

The foregoing and other features and advantages of the present invention will be more fully understood from the following detailed description of illustrative embodiments taken in conjunction with the accompanying drawings.

FIG. 1A and FIG. 1B depict the HDAC activity for HDACs 1-9 in the presence of various HDAC inhibitors, including compounds of Formula I.

FIG. 2 illustrates the activity of HDAC3 in the presence of various HDAC inhibitors.

FIG. 3 shows the selectivity of LW3 for HDAC3 inhibition in comparison to known HDAC inhibitors.

FIG. 4A, FIG. 4B, FIG. 4C, FIG. 4D, and FIG. 4E depict the inhibitory activity of the compounds disclosed herein for all HDAC isoforms.

### DETAILED DESCRIPTION

Provided herein are compounds, or pharmaceutically acceptable salts thereof, that are useful in the treatment of cancer or a neurodegenerative disease in an individual in need thereof.

In a non-limiting aspect, these compounds can inhibit histone deacetylases. In a particular embodiment, the compounds provided herein are considered HDAC3 inhibitors. As such, in one aspect, the compounds provided herein are useful in the treatment of cancer or a neurodegenerative disease in an individual by acting as a HDAC3 inhibitor.

### Definitions

Listed below are definitions of various terms used to describe the compounds and methods provided herein. These definitions apply to the terms as they are used throughout this specification and claims, unless otherwise limited in specific instances, either individually or as part of a larger group.

Unless defined otherwise, all technical and scientific terms used herein generally have the same meaning as commonly understood by one of ordinary skill in the art. Generally, the nomenclature used herein and the laboratory procedures in cell culture, molecular genetics, organic chemistry, and peptide chemistry are those well-known and commonly employed in the art.

As used herein, the articles “a” and “an” refer to one or to more than one (i.e., to at least one) of the grammatical object of the article. By way of example, “an element” means one element or more than one element. Furthermore, use of the term “including” as well as other forms, such as “include,” “includes,” and “included,” is not limiting.

As used herein, the term “about” will be understood by persons of ordinary skill in the art and will vary to some extent on the context in which it is used. As used herein when referring to a measurable value such as an amount, a temporal duration, and the like, the term “about” is meant to encompass variations of ±20% or ±10%, including ±5%, ±1%, and ±0.1% from the specified value, as such variations are appropriate to perform the disclosed methods.

The term “treat,” “treated,” “treating,” or “treatment” includes the diminishment or alleviation of at least one symptom associated or caused by the state, disorder or disease being treated. In certain embodiments, the treatment comprises bringing into contact with HDAC3 an effective amount of a compound of Formula I for conditions related to cancers, hemoglobinopathies, or myelodysplastic syndrome.

As used herein, the term “prevent” or “prevention” means no disorder or disease development if none had occurred, or no further disorder or disease development if there had already been development of the disorder or disease. Also considered is the ability of one to prevent some or all of the symptoms associated with the disorder or disease.

As used herein, the term “patient,” “individual,” or “subject” refers to a human or a non-human mammal. Non-human mammals include, for example, livestock and pets, such as ovine, bovine, porcine, canine, feline and marine mammals. Preferably, the patient, subject, or individual is human.

As used herein, the terms “effective amount,” “pharmaceutically effective amount,” and “therapeutically effective amount” refer to a nontoxic but sufficient amount of an agent to provide the desired biological result. That result may be reduction or alleviation of the signs, symptoms, or causes of a disease, or any other desired alteration of a biological system. An appropriate therapeutic amount in any individual case may be determined by one of ordinary skill in the art using routine experimentation.

As used herein, the term “pharmaceutically acceptable” refers to a material, such as a carrier or diluent, which does not abrogate the biological activity or properties of the compound, and is relatively non-toxic, i.e., the material may be administered to an individual without causing undesirable biological effects or interacting in a deleterious manner with any of the components of the composition in which it is contained.

As used herein, the term “pharmaceutically acceptable salt” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the compounds provided herein include the conventional non-toxic salts of the parent compound formed, for example, from non-toxic inorganic or organic acids. The pharmaceutically acceptable salts of the compounds provided herein can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. The phrase “pharmaceutically acceptable salt” is not limited to a mono, or 1:1, salt. For example, “pharmaceutically acceptable salt” also includes bis-salts, such as a bis-hydrochloride salt. Lists of suitable salts are found in Remington's Pharmaceutical Sciences, 17th ed., Mack Publishing Company, Easton, Pa., 1985, p. 1418 and Joumal of Pharmaceutical Science, 66, 2 (1977), each of which is incorporated herein by reference in its entirety.

As used herein, the term “composition” or “pharmaceutical composition” refers to a mixture of at least one compound provided herein with a pharmaceutically acceptable carrier. The pharmaceutical composition facilitates administration of the compound to a patient or subject. Multiple techniques of administering a compound exist in the art including, but not limited to, intravenous, oral, aerosol, parenteral, ophthalmic, pulmonary, and topical administration.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound provided herein or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound of Formula I, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations.

As used herein, “pharmaceutically acceptable carrier” also includes any and all coatings, antibacterial and antifungal agents, and absorption delaying agents, and the like that are compatible with the activity of the compound provided herein, and are physiologically acceptable to the patient. Supplementary active compounds may also be incorporated into the compositions. The “pharmaceutically acceptable carrier” may further include a pharmaceutically acceptable salt of the compound provided herein. Other additional ingredients that may be included in the pharmaceutical compositions provided herein are known in the art and described, for example, in Remington's Pharmaceutical Sciences (Genaro, Ed., Mack Publishing Co., 1985, Easton, Pa.), which is incorporated herein by reference.

An “oral dosage form” includes a unit dosage form prescribed or intended for oral administration. In an embodiment of the pharmaceutical combinations provided herein, the HDAC3 inhibitor (e.g., compounds of Formula I) is administered as an oral dosage form.

The term “HDAC” refers to histone deacetylases, which are enzymes that remove the acetyl groups from the lysine residues in core histones, thus leading to the formation of a condensed and transcriptionally silenced chromatin. There are currently 18 known histone deacetylases, which are classified into four groups. Class I HDACs, which include HDAC1, HDAC2, HDAC3, and HDAC8, are related to the yeast RPD3 gene. Class II HDACs, which include HDAC4, HDAC5, HDAC6, HDAC7, HDAC9, and HDAC10, are related to the yeast Hda1 gene. Class III HDACs, which are also known as the sirtuins are related to the Sir2 gene and include SIRT1-7. Class IV HDACs, which contains only HDAC11, has features of both Class I and II HDACs. The term “HDAC” refers to any one or more of the 18 known histone deacetylases, unless otherwise specified.

As used herein, the term “alkyl,” by itself or as part of another substituent means, unless otherwise stated, a straight or branched chain hydrocarbon having the number of carbon atoms designated (i.e., C₁-C₆-alkyl means an alkyl having one to six carbon atoms) and includes straight and branched chains. In an embodiment, C₁-C₆ alkyl groups are provided herein. Examples include methyl, ethyl, propyl, isopropyl, butyl, isobutyl, tert-butyl, pentyl, neopentyl, and hexyl. Other examples of C₁-C₆-alkyl include ethyl, methyl, isopropyl, isobutyl, n-pentyl, and n-hexyl.

As used herein, the term “alkoxy,” refers to the group —O-alkyl, wherein alkyl is as defined herein. Alkoxy includes, by way of example, methoxy, ethoxy, n-propoxy, isopropoxy, n-butoxy, sec-butoxy, t-butoxy and the like. In an embodiment, C₁-C₆ alkoxy groups are provided herein.

As used herein, the term “halo” or “halogen” alone or as part of another substituent means, unless otherwise stated, a fluorine (fluoro), chlorine (chloro), bromine (bromo), or iodine (iodo) atom, preferably, fluorine, chlorine, or bromine, more preferably, fluorine or chlorine.

As used herein, the term “cycloalkyl” means a non-aromatic carbocyclic system that is partially or fully saturated having 1, 2 or 3 rings wherein such rings may be fused. The term “fused” means that a second ring is present (i.e., attached or formed) by having two adjacent atoms in common (i.e., shared) with the first ring. Cycloalkyl also includes bicyclic structures that may be bridged or spirocyclic in nature with each individual ring within the bicycle varying from 3-8 atoms. The term “cycloalkyl” includes, but is not limited to, cyclopropyl, cyclobutyl, cyclopentyl, cyclohexyl, bicyclo[3.1.0]hexyl, spiro[3.3]heptanyl, and bicyclo[1.1.1]pentyl. In an embodiment, C₄-C₇ cycloalkyl groups are provided herein.

As used herein, the term “heterocycloalkyl” means a non-aromatic carbocyclic system containing 1, 2, 3 or 4 heteroatoms selected independently from N, O, and S and having 1, 2 or 3 rings wherein such rings may be fused, wherein fused is defined above. Heterocycloalkyl also includes bicyclic structures that may be bridged or spirocyclic in nature with each individual ring within the bicycle varying from 3-8 atoms, and containing 0, 1, or 2 N, O, or S atoms. The term “heterocycloalkyl” includes cyclic esters (i.e., lactones) and cyclic amides (i.e., lactams) and also specifically includes, but is not limited to, epoxidyl, oxetanyl, tetrahydrofuranyl, tetrahydropyranyl (i.e., oxanyl), pyranyl, dioxanyl, aziridinyl, azetidinyl, pyrrolidinyl, 2,5-dihydro-1H-pyrrolyl, oxazolidinyl, thiazolidinyl, piperidinyl, morpholinyl, piperazinyl, thiomorpholinyl, 1,3-oxazinanyl, 1,3-thiazinanyl, 2-azabicyclo[2.1.1]hexanyl, 5-azabicyclo[2.1.1]hexanyl, 6-azabicyclo[3.1.1] heptanyl, 2-azabicyclo[2.2.1]heptanyl, 3-azabicyclo[3.1.1]heptanyl, 2-azabicyclo[3.1.1]heptanyl, 3-azabicyclo[3.1.0]hexanyl, 2-azabicyclo[3.1.0]hexanyl, 3-azabicyclo[3.2.1]octanyl, 8-azabicyclo[3.2.1]octanyl, 3-oxa-7-azabicyclo[3.3.1]nonanyl, 3-oxa-9-azabicyclo[3.3.1]nonanyl, 2-oxa-5-azabicyclo[2.2.1]heptanyl, 6-oxa-3-azabicyclo[3.1.1]heptanyl, 2-azaspiro[3.3]heptanyl, 2-oxa-6-azaspiro[3.3]heptanyl, 2-oxaspiro[3.3]heptanyl, 2-oxaspiro[3.5]nonanyl, 3-oxaspiro[5.3]nonanyl, and 8-oxabicydo[3.2.1]octanyl. In an embodiment, C₃-C₇ heterocydoalkyl groups are provided herein wherein C₃-C₇ refers to the number of atoms in the heterocyclici ring (i.e., 3-7 membered heterocyclic ring).

As used herein, the term “aromatic” refers to a carbocycle or heterocycle with one or more polyunsaturated rings and having aromatic character, i.e., having (4n+2) delocalized π (pi) electrons, where n is an integer.

As used herein, the term “aryl” means an aromatic carbocyclic system containing 1, 2 or 3 rings, wherein such rings may be fused, wherein fused is defined above. If the rings are fused, one of the rings must be fully unsaturated and the fused ring(s) may be fully saturated, partially unsaturated or fully unsaturated. The term “aryl” includes, but is not limited to, phenyl, naphthyl, indanyl, and 1,2,3,4-tetrahydronaphthalenyl. In some embodiments, aryl groups have 6 carbon atoms. In some embodiments, aryl groups have from six to ten carbon atoms. In some embodiments, aryl groups have from six to sixteen carbon atoms. In an embodiment, C₅-C₇ aryl groups are provided herein.

As used herein, the term “heteroaryl” means an aromatic carbocyclic system containing 1, 2, 3, or 4 heteroatoms selected independently from N, O, and S and having 1, 2, or 3 rings wherein such rings may be fused, wherein fused is defined above. The term “heteroaryl” includes, but is not limited to, furanyl, thiophenyl, oxazolyl, thiazolyl, imidazolyl, pyrazolyl, triazolyl, tetrazolyl, isoxazolyl, isothiazolyl, oxadiazolyl, thiadiazolyl, pyrdinyl, pyridazinyl, pyrimidinyl, pyrazinyl, imidazo[1,2-a]pyridinyl, pyrazolo[1,5-a]pyridinyl, 5,6,7,8-tetrahydroisoquinolinyl, 5,6,7,8-tetrahydroquinolinyl, 6,7-dihydro-5H-cyclopenta[b]pyridinyl, 6,7-dihydro-5H-cyclopenta[c]pyridinyl, 1,4,5,6-tetrahydrocyclopenta[c]pyrazolyl, 2,4,5,6-tetrahydrocyclopenta[c]pyrazolyl, 5,6-dihydro-4H-pyrrolo[1,2-b]pyrazolyl, 6,7-dihydro-5H-pyrrolo[1,2-b][1,2,4]triazolyl, 5,6,7,8-tetrahydro-[1,2,4]triazolo[1,5-a]pyridinyl, 4,5,6,7-tetrahydropyrazolo[1,5-a]pyridinyl, 4,5,6,7-tetrahydro-1H-indazolyl and 4,5,6,7-tetrahydro-2H-indazolyl. In an embodiment, C₅-C₁₃ heteroaryl groups are provided herein, wherein C₅-C₁₃ refers to the number of atoms in the heteroaryl ring (i.e., 5-13 membered heteroaryl ring).

It is to be understood that if an aryl, heteroaryl, cycloalkyl, or heterocycloalkyl moiety may be bonded or otherwise attached to a designated moiety through differing ring atoms (i.e., shown or described without denotation of a specific point of attachment), then all possible points are intended, whether through a carbon atom or, for example, a trivalent nitrogen atom. For example, the term “pyridinyl” means 2-, 3- or 4-pyridinyl, the term “thienyl” means 2- or 3-thioenyl, and so forth.

As used herein, the term “linker” means an organic moiety that connects two parts of a compound. Linkers typically comprise a direct bond or an atom such as oxygen or sulfur, a unit such as NRa, C(O), C(O)NH, SO, SO₂, SO₂NH or a chain of atoms, such as substituted or unsubstituted alkyl, substituted or unsubstituted alkenyl, substituted or unsubstituted alkynyl, arylalkyl, arylalkenyl, arylalkynyl, heteroarylalkyl, heteroarylalkenyl, heteroaryl-alkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylhetero-arylalkyl, alkynylheteroaryl-alkenyl, alkynylheteroarylalkynyl, alkylheterocyclylalkyl, alkyl-heterocyclylalkenyl, alkyl-heterocyclylalkynyl, alkenylheterocyclylalkyl, alkenylheterocyclyl-alkenyl, alkenylhetero-cyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenyl-heteroaryl, alkynylhereroaryl, where one or more methylenes can be interrupted or terminated by O, S, S(O), SO₂, NR₈, C(O), substituted or unsubstituted aryl, substituted or unsubstituted heteroaryl, substituted or unsubstituted heterocyclic; where R₈ is hydrogen, acyl, aliphatic or substituted aliphatic. In one embodiment, the linker is between one to about forty atoms, preferably ten to about forty atoms, preferably between about twenty to about forty atoms, more preferably thirty to about forty atoms, and most preferably about thirty-five to about thirty-eight atoms. In some embodiments, the linker is a C(O)NH(alkyl) chain, an alkoxy chain, or a combination thereof.

As used herein, the term “substituted” means that an atom or group of atoms has replaced hydrogen as the substituent attached to another group.

As used herein, the term “optionally substituted” means that the referenced group may be substituted or unsubstituted. In one embodiment, the referenced group is optionally substituted with zero substituents, i.e., the referenced group is unsubstituted. In another embodiment, the referenced group is optionally substituted with one or more additional group(s) individually and independently selected from groups described herein.

### Compounds

In an aspect, provided herein are compounds of Formula I:

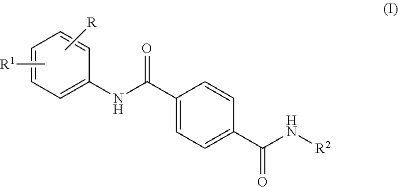

or a pharmaceutically acceptable salt thereof;

wherein:

R is selected from the group consisting of fluoro, bromo, chloro, —NH₂, —OH, —SH, —NHR³, —N(R³)₂, OR³, SR³, NO₂, thienyl, and CN;

R¹ is selected from the group consisting of fluoro, bromo, chloro, —NH₂, —OH, —SH, —NHR³, —N(R³)₂, OR³, SR³, NO₂, thienyl, and CN;

R² is selected from the group consisting of C₆-C₁₀ aryl, C₅-C₁₃ heteroaryl, C₃-C₁₀ cycloalkyl, C₃-C₁₀ heterocycloalkyl, C₁-C₆ alkyl-C₆-C₁₀ aryl, C₁-C₆ alkyl-C₅-C₁₃ heteroaryl, C₁-C₆ alkyl-C₃-C₁₀ cycloalkyl, C₁-C₆ alkyl-C₃-C₁₀ heterocycloalkyl, and -linker-biotin;

wherein C₅-C₁₃ heteroaryl, C₆-C₁₀ aryl, and C₁-C₆ alkyl are optionally substituted with one to three halo, phenyl, —C(O)Me, —OMe, methyl, NO₂, —SO₂Me, Ce heterocycloalkyl, C₅-C₆ heteroaryl, and CF₃; and

R³ is independently, at each occurrence, selected from the group consisting of H, C₁-C₆ alkyl, and C₁-C₆ alkoxy.

In an embodiment, R is selected from the group consisting of fluoro, bromo, chloro, —NH₂, —OH, and —SH; R¹ is selected from the group consisting of fluoro, bromo, chloro, —NH₂, —OH, and —SH; and R² is C₆-C₁₀ aryl or C₅-C₁₃ heteroaryl. In another embodiment, R is fluoro and R¹ is —NH₂. In yet another embodiment, R² is CH₂—C₅-C₁₃ heteroaryl. In still another embodiment, R² is quinoline. In an embodiment, R² is phenyl optionally substituted with one, two, or three morpholine, chloro, CF₃, OMe, Ph, and C(O)Me. In another embodiment, R² is indole. In yet another embodiment, R² is benzothiophene. In still another embodiment, R² is —CH₂-phenyl wherein phenyl optionally substituted with one, two, or three SO₂Me, NO₂, and bromo; and CH₂ is optionally substituted with methyl. In an embodiment, R² is benzothiazole optionally substituted with one, two, or three, bromo. In another embodiment, R² is pyrazole optionally substituted with methyl. In yet another embodiment, R² is carbazole. In still another embodiment, R² is piperadine. In an embodiment, R² is —CH₂CH₂-pyridine.

In still another embodiment, the compound of Formula I is a compound of Formula II:

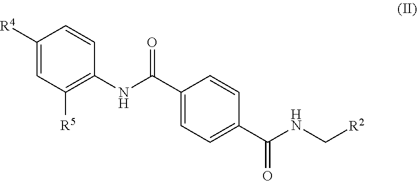

or a pharmaceutically acceptable salt thereof;

wherein:

R² is selected from the group consisting of C₆-C₁₀ aryl, C₅-C₁₀ heteroaryl, or linker-biotin, wherein C₆-C₁₀ aryl is optionally substituted with halo or SO₂Me;

R⁴ is selected from the group consisting of fluoro, bromo, chloro, —NH₂, —OH, and —SH; and

R⁵ is selected from the group consisting of fluoro, bromo, chloro, —NH₂, —OH, and —SH.

In an embodiment of Formula II, the linker has the following formula:

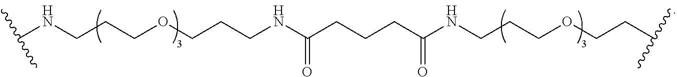

In an embodiment of Formula II, wherein R² is C₅-C₁₀ heteroaryl, R⁴ is fluoro, and R⁵ is

—NH₂. In another embodiment, R¹ is fluoro. In still another embodiment, R⁵ is NH₂. In an embodiment R² is pyridine. In another embodiment R is

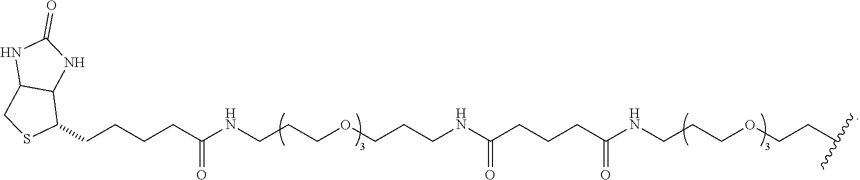

In an embodiment, the compound of Formula I or Formula II is a compound of Formula III:

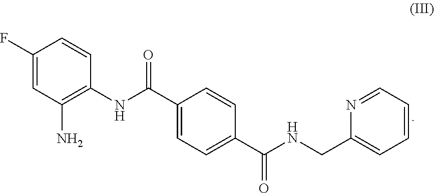 [SMILES: Nc1cc(F)ccc1NC(=O)c1ccc(C(=O)NCc2ccccn2)cc1]

or a pharmaceutically acceptable salt thereof.

In another embodiment, the compound of Formula I or Formula II is a compound of Formula IV:

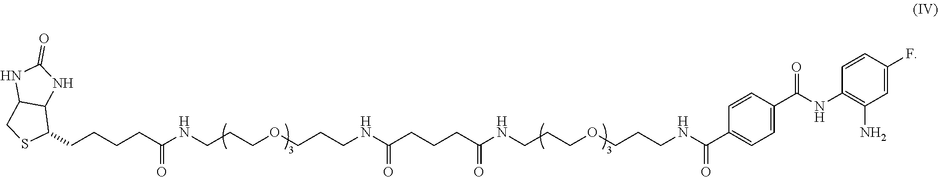

In another embodiment, a compound of Formula I is selected from a compound in Table 2 below:

TABLE 2

| Structure | Compound Name |
|---|---|
| 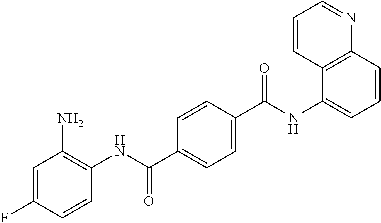 [SMILES: Nc1cc(F)ccc1NC(=O)c1ccc(C(=O)Nc2cccc3ncccc23)cc1] | DL-HDAC1 |
| 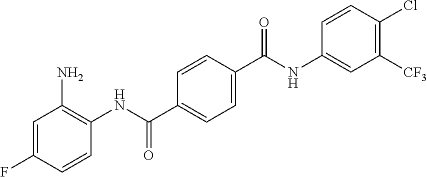 [SMILES: Nc1cc(F)ccc1NC(=O)c1ccc(C(=O)Nc2ccc(Cl)c(C(F)(F)F)c2)cc1] | DL-HDAC2 |
| 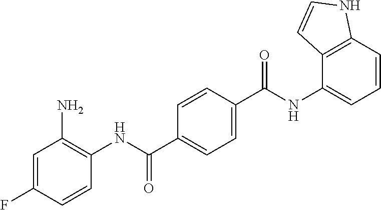 [SMILES: Nc1cc(F)ccc1NC(=O)c1ccc(C(=O)Nc2cccc3[nH]ccc23)cc1] | DL-HDAC3 |
| 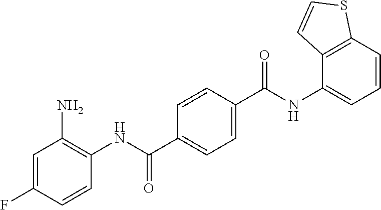 [SMILES: Nc1cc(F)ccc1NC(=O)c1ccc(C(=O)Nc2cccc3sccc23)cc1] | DL-HDAC4 |
| 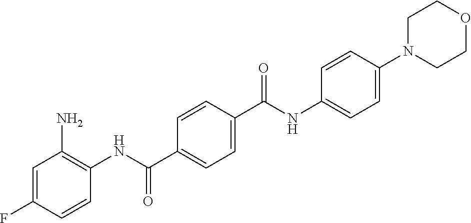 [SMILES: Nc1cc(F)ccc1NC(=O)c1ccc(C(=O)Nc2ccc(N3CCOCC3)cc2)cc1] | DL-HDAC5 |
| 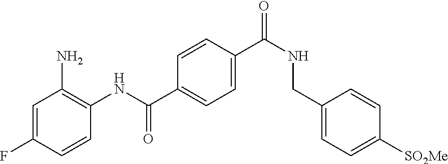 [SMILES: CS(=O)(=O)c1ccc(CNC(=O)c2ccc(C(=O)Nc3ccc(F)cc3N)cc2)cc1] | DL-HDAC6 |
| 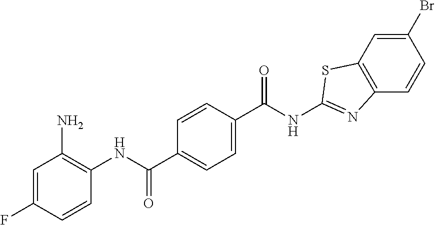 [SMILES: Nc1cc(F)ccc1NC(=O)c1ccc(C(=O)Nc2nc3ccc(Br)cc3s2)cc1] | DL-HDAC7 |
| 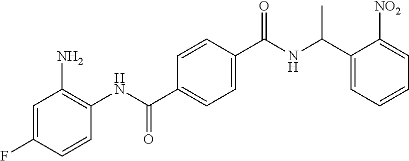 [SMILES: CC(NC(=O)c1ccc(C(=O)Nc2ccc(F)cc2N)cc1)c1ccccc1[N+](=O)[O-]] | DL-HDAC8 |
| 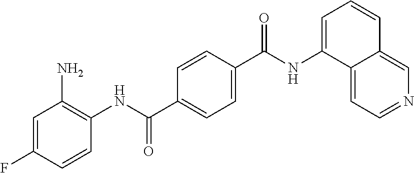 [SMILES: Nc1cc(F)ccc1NC(=O)c1ccc(C(=O)Nc2cccc3cnccc23)cc1] | DL-HDAC9 |
| 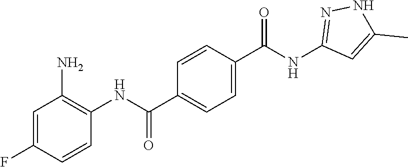 [SMILES: Cc1cc(NC(=O)c2ccc(C(=O)Nc3ccc(F)cc3N)cc2)n[nH]1] | DL-HDAC10 |
| 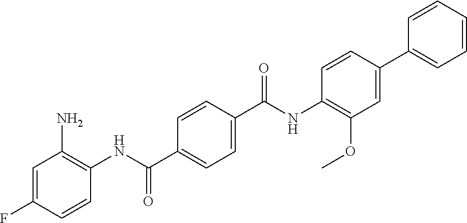 [SMILES: COc1cc(-c2ccccc2)ccc1NC(=O)c1ccc(C(=O)Nc2ccc(F)cc2N)cc1] | DL-HDAC11 |
| 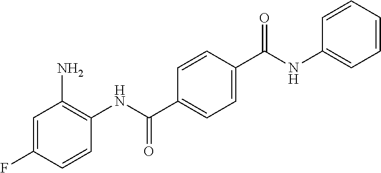 [SMILES: Nc1cc(F)ccc1NC(=O)c1ccc(C(=O)Nc2ccccc2)cc1] | DL-HDAC12 |
| 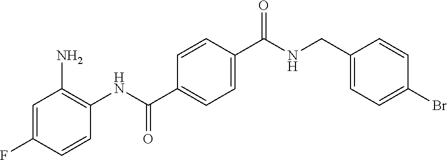 [SMILES: Nc1cc(F)ccc1NC(=O)c1ccc(C(=O)NCc2ccc(Br)cc2)cc1] | DL-HDAC13 |
| 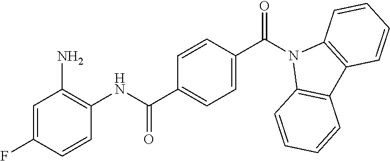 [SMILES: Nc1cc(F)ccc1NC(=O)c1ccc(C(=O)n2c3ccccc3c3ccccc32)cc1] | DL-HDAC14 |
| 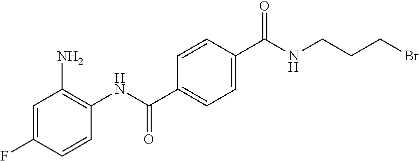 [SMILES: Nc1cc(F)ccc1NC(=O)c1ccc(C(=O)NCCCBr)cc1] | DL-HDAC15 |
| 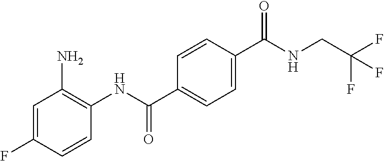 [SMILES: Nc1cc(F)ccc1NC(=O)c1ccc(C(=O)NCC(F)(F)F)cc1] | DL-HDAC16 |
| 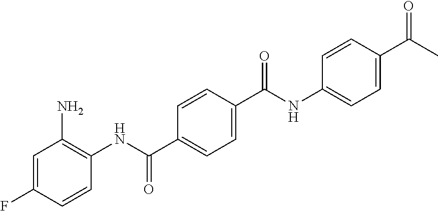 [SMILES: CC(=O)c1ccc(NC(=O)c2ccc(C(=O)Nc3ccc(F)cc3N)cc2)cc1] | DL-HDAC17 |
| 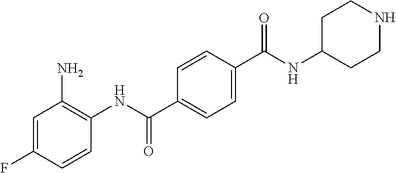 [SMILES: Nc1cc(F)ccc1NC(=O)c1ccc(C(=O)NC2CCNCC2)cc1] | DL-HDAC18 |
| 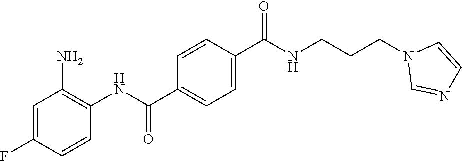 [SMILES: Nc1cc(F)ccc1NC(=O)c1ccc(C(=O)NCCCn2ccnc2)cc1] | DL-HDAC19 |
| 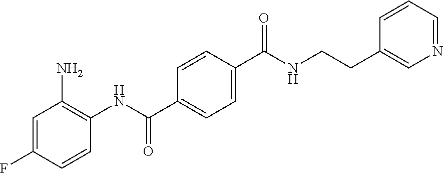 [SMILES: Nc1cc(F)ccc1NC(=O)c1ccc(C(=O)NCCc2cccnc2)cc1] | DL-HDAC20 |
| 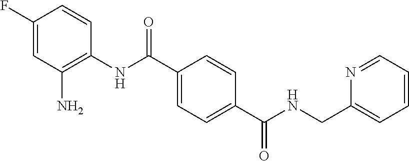 [SMILES: Nc1cc(F)ccc1NC(=O)c1ccc(C(=O)NCc2ccccn2)cc1] | LW3 |

or a pharmaceutically acceptable salt thereof.

In yet another aspect, provided herein are pharmaceutical compositions comprising any of the compounds described herein, or a pharmaceutically acceptable salt thereof, together with a pharmaceutically acceptable carrier.

In one embodiment, the disclosed compounds may exist as tautomers. All tautomers are included within the scope of the compounds presented herein.

Compounds described herein also include isotopically-labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In another embodiment, isotopically-labeled compounds are useful in drug or substrate tissue distribution studies. In another embodiment, substitution with heavier isotopes such as deuterium affords greater metabolic stability (for example, increased in vivo half-life or reduced dosage requirements). In yet another embodiment, the compounds described herein include a ²H (i.e., deuterium) isotope.

In still another embodiment, substitution with positron emitting isotopes, such as ¹¹C, ¹⁸F, ¹⁵O and ¹³N, is useful in Positron Emission Topography (PET) studies for examining substrate receptor occupancy. Isotopically-labeled compounds are prepared by any suitable method or by processes using an appropriate isotopically-labeled reagent in place of the non-labeled reagent otherwise employed.

The specific compounds described herein, and other compounds encompassed by one or more of the formulas described herein having different substituents are synthesized using techniques and materials described herein and as described, for example, in Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), Larock's Comprehensive Organic Transformations (VCH Publishers Inc., 1989). March, Advanced Organic Chemistry 40 Ed., (Wiley 1992); Carey and Sundberg, Advanced Organic Chemistry 4th Ed., Vols. A and B (Plenum 2000, 2001), and Green and Wuts, Protective Groups in Organic Synthesis 3rd Ed., (Wiley 1999) (all of which are incorporated by reference for such disclosure). General methods for the preparation of compounds as described herein are modified by the use of appropriate reagents and conditions, for the introduction of the various moieties found in the Formulas as provided herein.

Compounds described herein are synthesized using any suitable procedures starting from compounds that are available from commercial sources, or are prepared using procedures described herein.

### Methods of Treatment

The compounds and compositions provided herein can be used in a method of treating a disease or condition in a subject, said method comprising administering to the subject a compound provided herein, or a pharmaceutical composition comprising a compound provided herein.

In one aspect, provided herein is a method of selectively inhibiting HDAC3 over other HDACs (e.g., HDAC1, HDAC2, and HDAC6) in a subject, comprising administering to the subject a compound of Formula I, II, III or IV or pharmaceutically acceptable salts thereof.

In one embodiment, the compound of any of the formulae herein (e.g., Formula I or II) has a selectivity for HDAC3 of 5 to 1000 fold over other HDACs.

In another embodiment, the compound of any of the formulae herein (e.g., Formula I or II) has a selectivity for HDAC3 when tested in a HDAC enzyme assay of about 5 to 1000 fold over other HDACs.

In one aspect, provided herein are methods of inhibiting the activity of histone deacetylase 3 (HDAC3) in an individual in need thereof, comprising administering to the individual any of the compounds or compositions described herein.

In an aspect, provided herein are methods of treating a disease mediated by HDAC3 in an individual in need thereof, comprising administering to the individual any of the compounds or compositions described herein.

In another aspect, provided herein are methods of treating cancer in an individual in need thereof, comprising administering to the individual any of the compounds or compositions described herein.

In an embodiment, the cancer is Medulloblastoma, rhabdomyosarcoma, Hodgkin lymphoma, acute myeloid leukemia, myelodysplastic syndrome, pancreatic cancer, colon cancer, ovarian cancer, lung cancer, stomach cancer, a muscle cancer, a bone cancer, or a skin cancer. In another embodiment, the cancer is rhabdomyosarcoma. In yet another embodiment, the cancer is alveloar rhabdomyosarcoma. In still another embodiment, the cancer is pediatric rhabdomyosarcoma.

In certain embodiments, the cancer is lung cancer, colon and rectal cancer, breast cancer, prostate cancer, liver cancer, pancreatic cancer, brain cancer, kidney cancer, ovarian cancer, stomach cancer, skin cancer, bone cancer, gastric cancer, breast cancer, glioma, glioblastoma, neuroblastoma, hepatocellular carcinoma, papillary renal carcinoma, head and neck squamous cell carcinoma, leukemia, lymphomas, myelomas, retinoblastoma, cervical cancer, melanoma and/or skin cancer, bladder cancer, uterine cancer, testicular cancer, esophageal cancer, and solid tumors. In some embodiments, the cancer is lung cancer, colon cancer, breast cancer, neuroblastoma, leukemia, or lymphomas. In other embodiments, the cancer is lung cancer, colon cancer, breast cancer, neuroblastoma, leukemia, or lymphoma. In a further embodiment, the cancer is non-small cell lung cancer (NSCLC) or small cell lung cancer.

In further embodiments, the cancer is a hematologic cancer, such as leukemia or lymphoma. In a certain embodiment, lymphoma is Hodgkin's lymphoma or Non-Hodgkin's lymphoma. In certain embodiments, leukemia is myeloid, lymphocytic, myelocytic, lymphoblastic, or megakaryotic leukemia.

In a particular embodiment, the leukemia is acute myelogenous leukemia and megakaryocytic leukemia.

In an embodiment, the subject is human.

In yet another aspect, provided herein are methods of treating a neurodegenerative disease in an individual in need thereof, comprising administering to the individual a compound of Formula I or a pharmaceutically acceptable salt thereof.

In an embodiment, the neurodegenerative disease is Spinal Muscular Atrophy, polyglutamine-related diseases, or amyotrophic lateral sclerosis. In another embodiment, polyglutamine-related disease is Huntington disease, dentatorubral-pallidoluysian atrophy, or spinocerebellar ataxia type 6 (SCA6).

Thus, in another aspect, methods for the treatment of a disease mediated by HDAC3 are provided comprising administering a therapeutically effective amount of a compound of Formula I, as described herein, to an individual in need thereof. In certain embodiments, the individual is identified as in need of such treatment. In certain embodiments, a method for the treatment of a diseases is provided comprising administering a therapeutically effective amount of a compound of Formula I, or a pharmaceutical composition comprising a compound of Formula I to a subject in need thereof, in such amounts and for such time as is necessary to achieve the desired result.

In certain embodiments, the method involves the administration of a therapeutically effective amount of a compound of Formula I or a pharmaceutically acceptable derivative thereof to a subject (including, but not limited to a human or animal) in need of it (including a subject identified as in need).

Thus, in a further aspect, methods for the treatment of a disease mediated by HDAC3 are provided comprising administering a therapeutically effective amount of a compound of Formula I, as described herein, to a subject in need thereof. In certain embodiments, the subject is identified as in need of such treatment. In certain embodiments, a method for the treatment of a disease is provided comprising administering a therapeutically effective amount of a compound of Formula I, or a pharmaceutical composition comprising a compound of Formula II to a subject in need thereof, in such amounts and for such time as is necessary to achieve the desired result.

In certain embodiments, the method involves the administration of a therapeutically effective amount of a compound of Formula II, or a pharmaceutically acceptable derivative thereof to a subject (including, but not limited to a human or animal) in need of it (including a subject identified as in need).

### Admonition/Dosage/Formulations

In another aspect, provided herein is a pharmaceutical composition comprising at least one compound provided herein, together with a pharmaceutically acceptable carrier.

Actual dosage levels of the active ingredients in the pharmaceutical compositions provided herein may be varied so as to obtain an amount of the active ingredient that is effective to achieve the desired therapeutic response for a particular patient, composition, and mode of administration, without being toxic to the patient.

In particular, the selected dosage level will depend upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could begin administration of the pharmaceutical composition to dose the disclosed compound at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of the disclosed compound calculated to produce the desired therapeutic effect in association with the required pharmaceutical vehicle. The dosage unit forms of the compounds provided herein are dictated by and directly dependent on (a) the unique characteristics of the disclosed compound and the particular therapeutic effect to be achieved, and (b) the limitations inherent in the art of compounding/formulating such a disclosed compound for the treatment of pain, a depressive disorder, or drug addiction in a patient.

In one embodiment, the compounds provided herein are formulated using one or more pharmaceutically acceptable excipients or carriers. In one embodiment, the pharmaceutical compositions provided herein comprise a therapeutically effective amount of a disclosed compound and a pharmaceutically acceptable carrier.

Routes of administration of any of the compositions disclosed herein include oral, nasal, rectal, intravaginal, parenteral, buccal, sublingual or topical. The compounds disclosed herein may be formulated for administration by any suitable route, such as for oral or parenteral, for example, transdermal, transmucosal (e.g., sublingual, lingual, (trans)buccal, (trans)urethral, vaginal (e.g., trans- and perivaginally), (intra)nasal and (trans)rectal), intravesical, intrapulmonary, intraduodenal, intragastrical, intrathecal, subcutaneous, intramuscular, intradermal, intra-arterial, intravenous, intrabronchial, inhalation, and topical administration. In one embodiment, the preferred route of administration is oral.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present invention are not limited to the particular formulations and compositions that are described herein.

For oral application, particularly suitable are tablets, dragees, liquids, drops, suppositories, or capsules, caplets and gel caps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For parenteral administration, the disclosed compounds may be formulated for injection or infusion, for example, intravenous, intramuscular or subcutaneous injection or infusion, or for administration in a bolus dose or continuous infusion. Suspensions, solutions or emulsions in an oily or aqueous vehicle, optionally containing other formulatory agents such as suspending, stabilizing or dispersing agents may be used.

Those skilled in the art will recognize, or be able to ascertain using no more than routine experimentation, numerous equivalents to the specific procedures, embodiments, claims, and examples described herein. Such equivalents were considered to be within the scope of this disclosure and covered by the claims appended hereto. For example, it should be understood, that modifications in reaction conditions, including but not limited to reaction times, reaction size/volume, and experimental reagents, such as solvents, catalysts, pressures, atmospheric conditions, e.g., nitrogen atmosphere, and reducing/oxidizing agents, with art-recognized alternatives and using no more than routine experimentation, are within the scope of the present application.

It is to be understood that wherever values and ranges are provided herein, all values and ranges encompassed by these values and ranges, are meant to be encompassed within the scope of the present disclosure. Moreover, all values that fall within these ranges, as well as the upper or lower limits of a range of values, are also contemplated by the present application.

The following examples further illustrate aspects of the present invention. However, they are in no way a limitation of the teachings or disclosure of the present invention as set forth.

### Processes

In one aspect, provided herein are processes for preparing a compound of Formula V:

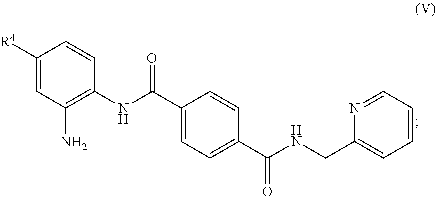

comprising reacting a compound of Formula VI:

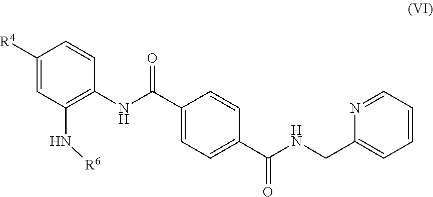

with an acid in a solvent;

wherein

R⁴ is selected from the group consisting of fluoro, bromo, and chloro; and

R⁶ is a protecting group selected from the group consisting of acetyl (Ac), benzyl (Bn), tert-butyloxycarbonyl (Boc), benzoyl (Bz), carboxybenzyl (Cbz), carbamate, 3,4-dimethoxy-benzyl (DMPM), 9-fluorenylmethyloxycarbonyl (Fmoc), p-methoxybenzyl carbonyl (Moz), 4-nitrobenzylsulfonyl (Nos), p-methoxybenzyl (PMB), p-methoxyphenyl (PMP), 4-toluenesulfonyl (Tos), and trichloroethyl chloroformate (Troc).

In an embodiment, the acid is hydrochloric acid. In another embodiment, the solvent is dioxane. In yet another embodiment, R⁶ is tert-butyloxycarbonyl (Boc). In still another embodiment, R⁴ is fluoro.

In another aspect, provided herein are processes for preparing a compound of Formula VI:

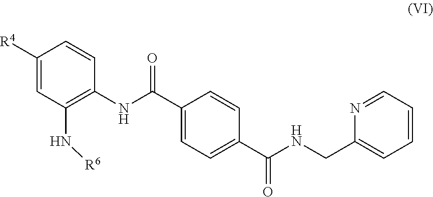

comprising reacting a compound of Formula VII:

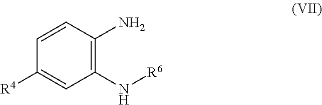

with a compound of Formula VIII:

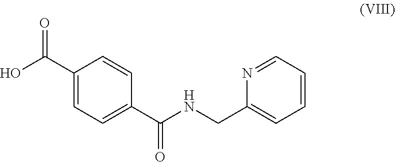 [SMILES: O=C(O)c1ccc(C(=O)NCc2ccccn2)cc1]

in the presence of a peptide coupling reagent, a base, and a solvent;

wherein

R⁴ is selected from the group consisting of fluoro, bromo, and chloro; and

R⁶ is a protecting group selected from the group consisting of acetyl (Ac), benzyl (Bn), tert-butyloxycarbonyl (Boc), benzoyl (Bz), carboxybenzyl (Cbz), carbamate, 3,4-dimethoxy-benzyl (DMPM), 9-fluorenylmethyloxycarbonyl (Fmoc), p-methoxybenzyl carbonyl (Moz), 4-nitrobenzylsulfonyl (Nos), p-methoxybenzyl (PMB), p-methoxyphenyl (PMP), 4-toluenesulfonyl (Tos), and trichloroethyl chloroformate (Troc).

In an embodiment, the peptide coupling reagent is 1-[bis(dimethylamino)-methylene]-1H-1,2,3-triazolo[4,5-b]pyridinium 3-oxid hexafluorophosphate (HATU). In another embodiment, the base is N,N-diisopropylethyl amine (DIPEA or Hünig's base). In yet another embodiment, the solvent is dimethylformamide. In still another embodiment, R⁶ is tert-butyloxycarbonyl (Boc). In an embodiment, R⁴ is fluoro.

In yet another aspect, provided herein are processes for preparing a compound of Formula VII:

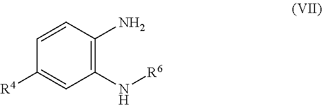

comprising treating a compound of Formula IX:

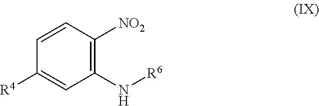

with hydrogen gas in the presence of a palladium catalyst and a solvent or mixture of solvents;

wherein

R⁴ is selected from the group consisting of fluoro, bromo, and chloro; and

R⁶ is a protecting group selected from the group consisting of acetyl (Ac), benzyl (Bn), tert-butyloxycarbonyl (Boc), benzoyl (Bz), carboxybenzyl (Cbz), carbamate, 3,4-dimethoxy-benzyl (DMPM), 9-fluorenylmethyloxycarbonyl (Fmoc), p-methoxybenzyl carbonyl (Moz), 4-nitrobenzylsulfonyl (Nos), p-methoxybenzyl (PMB), p-methoxyphenyl (PMP), 4-toluenesulfonyl (Tos), and trichloroethyl chloroformate (Troc).

In an embodiment, the palladium catalyst in 10% palladium on carbon. In another embodiment, the solvent is a mixture of ethanol and ethyl acetate. In yet another embodiment, R⁴ is fluoro. In still another embodiment, R⁶ is tert-butyloxycarbonyl (Boc).

In still another aspect, provided herein are processes for preparing a compound of Formula IX:

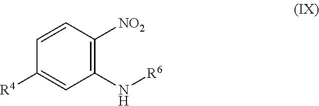

comprising reacting a compound of Formula X:

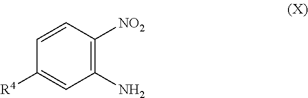

with a protecting group reagent and a base, or combination of bases, in a solvent;

wherein

R⁴ is selected from the group consisting of fluoro, bromo, and chloro; and

R⁶ is a protecting group selected from the group consisting of acetyl (Ac), benzyl (Bn), tert-butyloxycarbonyl (Boc), benzoyl (Bz), carboxybenzyl (Cbz), carbamate, 3,4-dimethoxy-benzyl (DMPM), 9-fluorenylmethyloxycarbonyl (Fmoc), p-methoxybenzyl carbonyl (Moz), 4-nitrobenzylsulfonyl (Nos), p-methoxybenzyl (PMB), p-methoxyphenyl (PMP), 4-toluenesulfonyl (Tos), and trichloroethyl chloroformate (Troc).

In an embodiment, the protecting group reagent is di-tert-butyl dicarbonate (Boc₂O) and R⁶ is tert-butyloxycarbonyl (Boc). In another embodiment, the base is a combination of 4-dimethylaminopyridine (DMAP) and is N,N-diisopropylethyl amine (DIPEA or Hünig's base). In yet another embodiment, the solvent is dichloromethane (DCM). In still another embodiment, R⁴ is fluoro.

In an aspect, provided herein are processes for preparing a compound of Formula VIII:

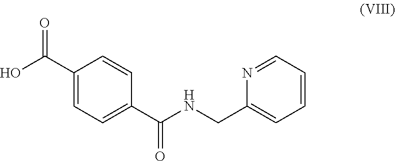 [SMILES: O=C(O)c1ccc(C(=O)NCc2ccccn2)cc1]

comprising reacting a compound of Formula XI:

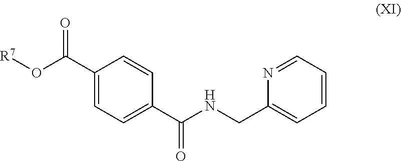

with a base in a solvent;

wherein R⁷ is C₁-C₆ alkyl.

In an embodiment, the base in sodium hydroxide (NaOH). In another embodiment, the solvent is methanol. In yet another embodiment, R⁷ is —CH₃.

In another aspect, provided herein are processes for preparing a compound of Formula XI:

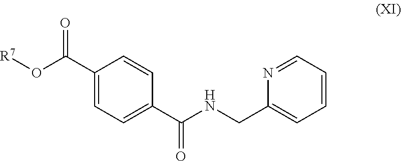

comprising reacting a compound of Formula XII:

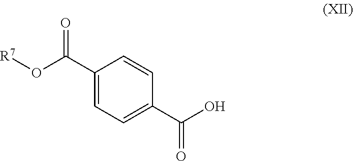

with a compound of Formula XIII:

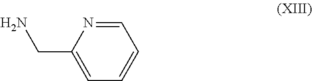

in the presence of a peptide coupling reagent, a base, and a solvent;

wherein R⁷ is C₁-C₆ alkyl.

In an embodiment, the peptide coupling reagent is 1-[bis(dimethylamino)-methylene]-1H-1,2,3-triazolo[4,5-b]pyridinium 3-oxid hexafluorophosphate (HATU). In another embodiment, the base is N,N-diisopropylethylamine (DIPEA or Hunig's base). In yet another embodiment, the solvent is dimethylformamide (DMF). In still another embodiment, R⁷ is —CH₃.

In yet another aspect, provided herein are processes for preparing a compound of Formula IV:

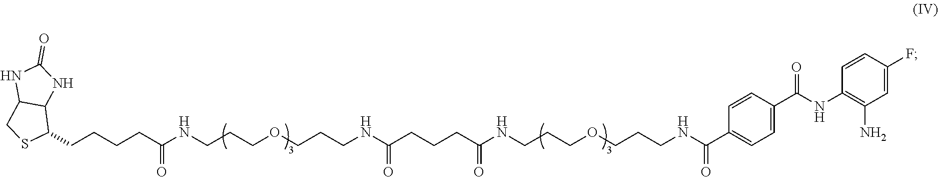

comprising reacting a compound of Formula XIV:

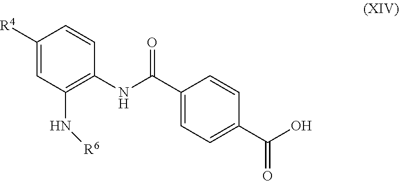

with a compound of Formula XV:

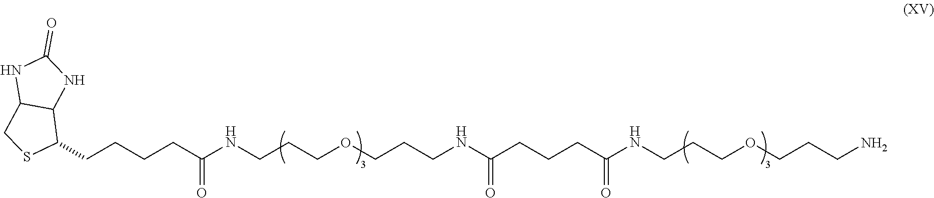

in the presence of a peptide coupling reagent and a base in a solvent, then further reacting the product of the above reaction with an acid;

wherein

R⁴ is selected from the group consisting of fluoro, bromo, and chloro; and

R⁶ is a protecting group selected from the group consisting of acetyl (Ac), benzyl (Bn), tert-butyloxycarbonyl (Boc), benzoyl (Bz), carboxybenzyl (Cbz), carbamate, 3,4-dimethoxy-benzyl (DMPM), 9-fluorenylmethyloxycarbonyl (Fmoc), p-methoxybenzyl carbonyl (Moz), 4-nitrobenzylsulfonyl (Nos), p-methoxybenzyl (PMB), p-methoxyphenyl (PMP), 4-toluenesulfonyl (Tos), and trichloroethyl chloroformate (Troc).

In an embodiment, the peptide coupling reagent is 1-[bis(dimethylamino)-methylene]-1H-1,2,3-triazolo[4,5-b]pyridinium 3-oxide hexafluorophosphate (HATU). In another embodiment, the base is N,N-diisopropylethylamine (DIPEA or Hunig's base). In yet another embodiment, the solvent is dimethylformamide (DMF). In still another embodiment, the acid is hydrochloric acid (HCl). In an embodiment, R⁴ is fluoro and R⁶ is tert-butyloxycarbonyl (Boc).

In yet another aspect, provided herein are processes for preparing a compound of Formula XIV:

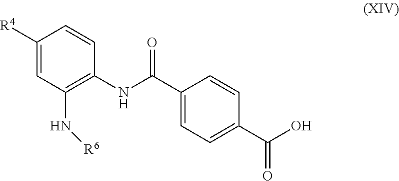

comprising reacting a compound of Formula XVI:

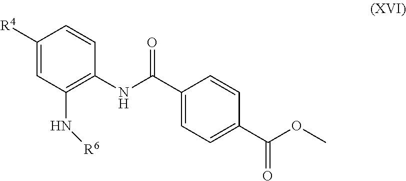

with a base in a solvent;

wherein

R⁴ is selected from the group consisting of fluoro, bromo, and chloro; and

R⁶ is a protecting group selected from the group consisting of acetyl (Ac), benzyl (Bn), tert-butyloxycarbonyl (Boc), benzoyl (Bz), carboxybenzyl (Cbz), carbamate, 3,4-dimethoxy-benzyl (DMPM), 9-fluorenylmethyloxycarbonyl (Fmoc), p-methoxybenzyl carbonyl (Moz), 4-nitrobenzylsulfonyl (Nos), p-methoxybenzyl (PMB), p-methoxyphenyl (PMP), 4-toluenesulfonyl (Tos), and trichloroethyl chloroformate (Troc).

In an embodiment, the base in sodium hydroxide (NaOH). In another embodiment, the solvent is methanol (MeOH). In yet another embodiment, R⁴ is fluoro and R⁶ is tert-butyloxycarbonyl (Boc).

In still another aspect, provided herein are processes for preparing a compound of Formula XVI:

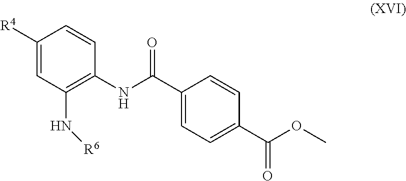

comprising reacting a compound of Formula XVII:

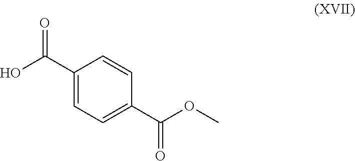

with a compound of Formula VII:

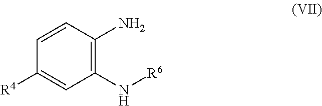

in the presence of a peptide coupling reagent and a base in a solvent;

wherein

R⁴ is selected from the group consisting of fluoro, bromo, and chloro; and

R⁶ is a protecting group selected from the group consisting of acetyl (Ac), benzyl (Bn), tert-butyloxycarbonyl (Boc), benzoyl (Bz), carboxybenzyl (Cbz), carbamate, 3,4-dimethoxy-benzyl (DMPM), 9-fluorenylmethyloxycarbonyl (Fmoc), p-methoxybenzyl carbonyl (Moz), 4-nitrobenzylsulfonyl (Nos), p-methoxybenzyl (PMB), p-methoxyphenyl (PMP), 4-toluenesulfonyl (Tos), and trichloroethyl chloroformate (Troc).

In an embodiment, the peptide coupling reagent is 1-[bis(dimethylamino)-methylene]-1H-1,2,3-triazolo[4,5-b]pyridinium 3-oxide hexafluorophosphate (HATU). In another embodiment, the base is N,N-diisopropylethylamine (DIPEA or Hünig's base). In yet another embodiment, the solvent is dimethylformamide (DMF). In still another embodiment, R⁴ is fluoro and R⁶ is tert-butyloxycarbonyl (Boc).

### EXAMPLES

The invention is further illustrated by the following examples, which should not be construed as further limiting. The practice of the present invention will employ, unless otherwise indicated, conventional techniques of organic synthesis, cell biology, cell culture, molecular biology, transgenic biology, microbiology and immunology, which are within the skill of the art.

### Abbreviations

° C. degree Celsius
DIPEA N,N-Diisopropylethylamine
DMAP 4-dimethylaminopyridine
DMF dimethylformamide
EA/EtOAc ethyl acetate
HATU O-(7-Azabenzotriazol-1-yl)-N,N,N′,N′-tetramethyluronium Hexafluorophosphate
HPLC high performance liquid chromatography
MeOH methanol
MeCN acetonitrile
PE petroleum ether
Ph phenyl
TFA trifluoroacetic acid
THF tetrahydrofuran
TLC thin layer chromatography

### Example 1—General Synthesis for Compounds of Formula I

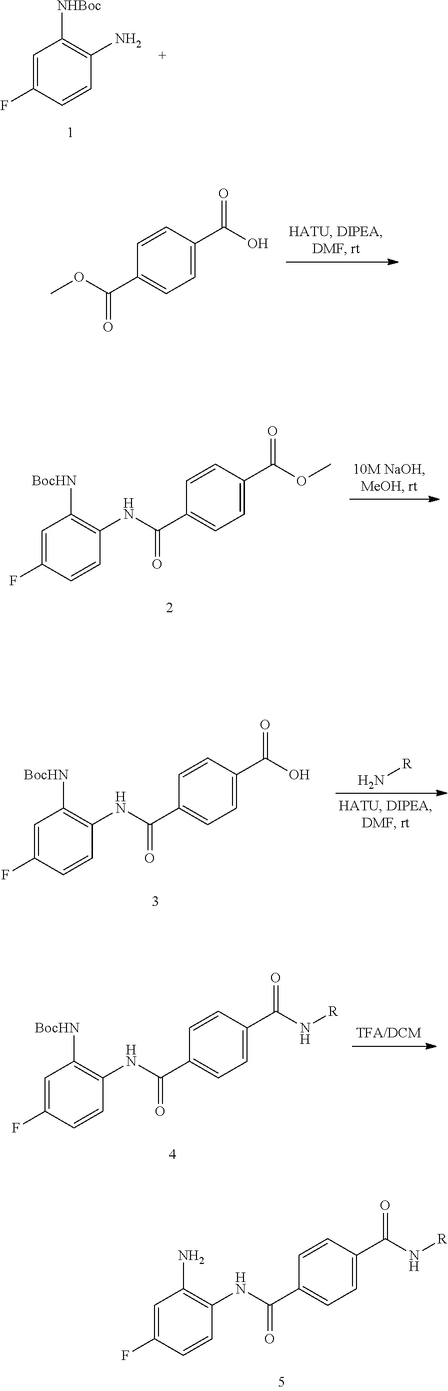

Step 1:

To a 100 mL round-bottom flask were added Mono-methyl terephthalate (795 mg, 4.4 mmol, 1 eq.) and HATU (2.5 g, 6.6 mmol, 1.5 eq.). Then DMF (20 mL) was added to generate a colorless solution, then DIPEA (1.1 mL, 1.5 eq.) was added and the resulting mixture was stirred at room temperature for 30 min. After that, compound 1 (1.0 g, 4.4 mmol, 1 eq.) was added into the reaction flask. The reaction was continuously stirred at room temperature for 16 hours. After that, the reaction mixture was worked-up by adding 100 mL water, then extracted with ethyl acetate (20 mL×3). The organic layers were combined and dried over MgSO₄, then condensed to get the residue. The residue was purified via ISCO (ethyl acetate/hexane=1/20˜1/3) to give compound 2 as white powder 1.25 g, yield: 58.5%. MS (ESI) calcd. for C₂₀H₂₁FN₂O₅: 388.40, Found: 289.12, 333.09, [M+1] 389.19. [2M+1] 777.29.

Step 2:

Compound 2 (1.25 g, 3.2 mmol) was added to MeOH (10 mL) to get a suspension. Then NaOH (aq. 4M, 4 mL) was added and the result reaction mixture was allowed to stir at room temperature for 30 minutes. After that, the mixture became a clean solution, the reaction was continued to stir for another 8 hours. After that, MeOH was removed under reduced pressure and the aqueous was adjusted to pH 5 by adding 5M HCl solution. After the pH reached 6, salts dissolved out and the mixture get filtered directly to get the compound 3 after dried under reduced pressure as lighter yellow powder 1.2 g, yield: 99%. MS (ESI) calcd. for C₁₉H₁₉FN₂O₅: 374.37, Found: 275.10, 319.11, [M+1] 375.17, [2M+1] 749.25. ¹H NMR (500 MHz, DMSO-d₆) δ 9.91 (s, 1H), 8.83 (s, 1H), 8.07 (s, 4H), 7.56 (dd, J=11.2, 3.0 Hz, 1H), 7.46 (dd, J=8.9, 6.2 Hz, 1H), 6.97 (td, J=8.4, 3.0 Hz, 1H), 1.45 (s, 9H).

Step 3:

To an 8 mL flask were added compound 3 (10 mg, 0.027 mmol, 1 eq.) and HATU (20.5 mg, 0.054 mmol, 2.0 eq.). Then DMF (0.5 mL) was added to generate a colorless solution, then DIPEA (9 μL, 2.0 eq.) was added and the resulting mixture was stirred at room temperature for 30 min. After that, an amine (0.027 mmol, 1 eq.) was added into the reaction flask. The reaction was continue stirred at room temperature for 16 hours. After that, the reaction mixture was purified directly via Pre-HPLC (MeCN/Water, 0.1% formic acid) to give compound 4 as white or yellow powder after lyophilization. Yield: 30-72%

Step 4:

To an 8 mL flask were added compound 4 (1 eq.) and TFA (200 μL in 1 mL DCM) The reaction was stirred at room temperature for 16 hours. After that, the reaction mixture was purified directly via Pre-HPLC (MeCN/water, 0.1% formic acid) to give 5 as white or yellow powder after lyophilization. Yield, >85%.

The following compounds were prepared using the method described above.

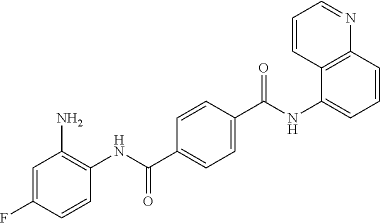 [SMILES: Nc1cc(F)ccc1NC(=O)c1ccc(C(=O)Nc2cccc3ncccc23)cc1]

Compound DL-HDAC1. 2.5 mg, yield: 93.2%; yellow powder. ¹H NMR (500 MHz, DMSO-d₆) δ 10.70 (s, 1H), 9.79 (s, 1H), 8.96 (dd, J=4.1, 1.7 Hz, 1H), 8.43 (dt, J=8.5, 1.5 Hz, 1H), 8.25-8.14 (m, 4H), 7.99 (d, J=8.4 Hz, 1H), 7.83 (dd, J=8.5, 7.4 Hz, 1H), 7.74 (d, J=7.4 Hz, 1H), 7.59 (dd, J=8.6, 4.1 Hz, 1H), 7.16 (dd, J=8.6, 6.3 Hz, 1H), 6.57 (dd, J=11.2, 2.8 Hz, 1H), 6.39 (td, J=8.5, 2.9 Hz, 1H), 5.30 (s, 2H). MS (ESI) calcd. for C₂₃H₁₇FN₄O₂: 400.41, Found: [M+1] 400.91, 401.70, 402.31.

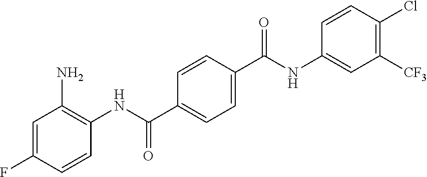 [SMILES: Nc1cc(F)ccc1NC(=O)c1ccc(C(=O)Nc2ccc(Cl)c(C(F)(F)F)c2)cc1]

Compound DL-HDAC2. 1.2 mg, yield: 90%; white powder. MS (ESI) calcd. for C₂₁H₁₄ClF₄N₃O₂: 451.81, Found: [M+1] 452.59, 453.27, 454.22.

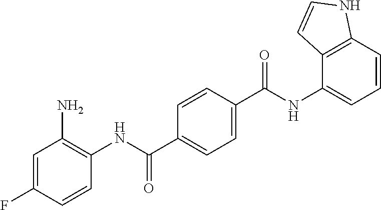 [SMILES: Nc1cc(F)ccc1NC(=O)c1ccc(C(=O)Nc2cccc3[nH]ccc23)cc1]

Compound DL-HDAC3. 1.5 mg, yield: 87%; yellow powder. MS (ESI) calcd. for C₂₂H₁₇FN₄O₂: 388.40, Found: [M+1] 389.30, 390.29.

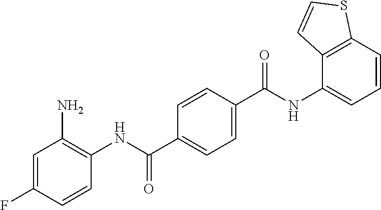 [SMILES: Nc1cc(F)ccc1NC(=O)c1ccc(C(=O)Nc2cccc3sccc23)cc1]

Compound DL-HDAC4. 0.9 mg, yield: 89%; white powder. MS (ESI) calcd. for C₂₂H₁₇FN₃O₂S: 405.45, Found: [M+1] 406.25, 407.28.

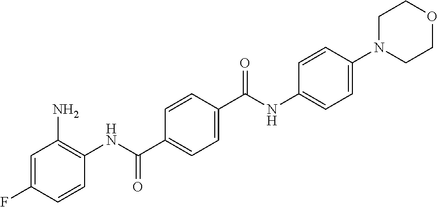 [SMILES: Nc1cc(F)ccc1NC(=O)c1ccc(C(=O)Nc2ccc(N3CCOCC3)cc2)cc1]

Compound DL-HDAC5. 5.2 mg, yield: 90%; yellow powder. ¹H NMR (500 MHz, DMSO-d₆) δ 10.21 (s, 1H), 9.74 (s, 1H), 8.13-8.02 (m, 4H), 7.67 (s, 2H), 7.16-7.12 (m, 1H), 6.99-6.94 (m, 2H), 6.56 (dd, J=11.3, 3.0 Hz, 1H), 6.38 (td, J=8.5, 2.9 Hz, 1H), 5.28 (s, 2H), 3.80-3.73 (m, 4H), 3.14-3.05 (m, 4H). MS (ESI) calcd. for C24H23FN4O3: 434.47, Found: [M+1] 435.21.

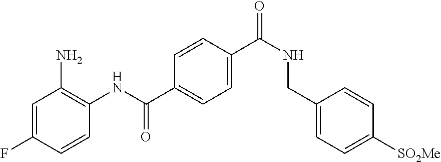 [SMILES: CS(=O)(=O)c1ccc(CNC(=O)c2ccc(C(=O)Nc3ccc(F)cc3N)cc2)cc1]

Compound DL-HDAC6. 4.6 mg, yield: 93%; yellow powder. ¹H NMR (500 MHz, DMSO-d₆) δ 10.27 (s, 1H), 9.74 (s, 1H), 8.18-8.04 (m, 4H), 7.75-7.68 (m, 2H), 7.48-7.45 (m, 1H), 7.43-7.38 (m, 1H), 7.36-7.32 (m, 1H), 7.14 (dd, J=8.6, 6.3 Hz, 1H), 7.07-6.99 (m, 1H), 5.28 (s, 2H), 5.11 (s, 2H). MS (ESI) calcd. for C₂₂H₂₀FN₃O₄S: 441.48, Found: 441.86, [M+1] 442.55, 443.23.

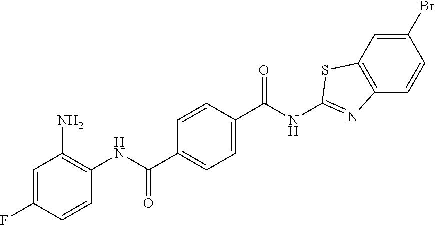 [SMILES: Nc1cc(F)ccc1NC(=O)c1ccc(C(=O)Nc2nc3ccc(Br)cc3s2)cc1]

Compound DL-HDAC7. 0.8 mg, yield: 90%; yellow powder. MS (ESI) calcd. for C₂₁H₁₄BrFN₄O₂S: 485.33, Found: 485.07, [M+1] 487.01.

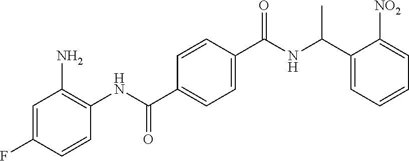 [SMILES: CC(NC(=O)c1ccc(C(=O)Nc2ccc(F)cc2N)cc1)c1ccccc1[N+](=O)[O-]]

Compound DL-HDAC8. 0.9 mg, yield: 92%; yellow powder. MS (ESI) calcd. for C₂₂H₁₉FN₄O₄: 422.42, Found: [M+1] 423.20, 424.23.

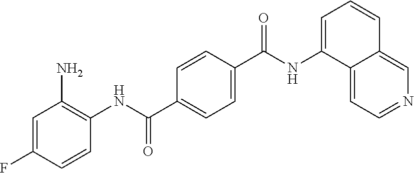 [SMILES: Nc1cc(F)ccc1NC(=O)c1ccc(C(=O)Nc2cccc3cnccc23)cc1]

Compound DL-HDAC9. 1.3 mg, yield: 85%; yellow powder. MS (ESI) calcd. for C₂₃H₁₇FN₄O₂: 400.41, Found: [M+1] 401.20.

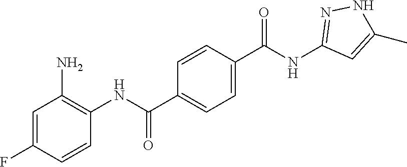 [SMILES: Cc1cc(NC(=O)c2ccc(C(=O)Nc3ccc(F)cc3N)cc2)n[nH]1]

Compound DL-HDAC10. 1.6 mg, yield: 86%; yellow powder. MS (ESI) calcd. for C₁₈H₁₆FN₅O₂: 353.36, Found: [M+1] 354.19, 355.21.

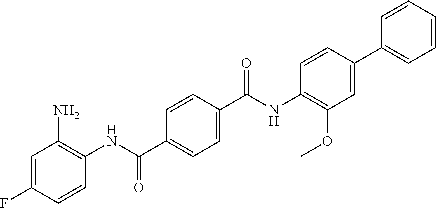 [SMILES: COc1cc(-c2ccccc2)ccc1NC(=O)c1ccc(C(=O)Nc2ccc(F)cc2N)cc1]

Compound DL-HDAC11. 0.9 mg, yield: 87%; yellow powder. MS (ESI) calcd. for C₂₇H₂₂FN₃O₃: 455.49, Found: 455.88, [M+1] 456.57, 457.21.

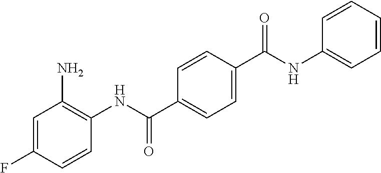 [SMILES: Nc1cc(F)ccc1NC(=O)c1ccc(C(=O)Nc2ccccc2)cc1]

Compound DL-HDAC12. 4.2 mg, yield: 92%; yellow powder. ¹H NMR (500 MHz, DMSO-d₆) δ 10.38 (s, 1H), 9.75 (s, 1H), 8.15-8.06 (m, 4H), 7.84-7.77 (m, 2H), 7.41-7.34 (m, 2H), 7.14 (tdd, J=7.4, 4.0, 2.0 Hz, 2H), 6.56 (dd, J=11.2, 2.9 Hz, 1H), 6.38 (td, J=8.5, 2.9 Hz, 1H), 5.28 (s, 2H). MS (ESI) calcd. for C₂₀H₁₆FN₃O₂: 349.37, Found: [M+1] 350.46, 351.22.

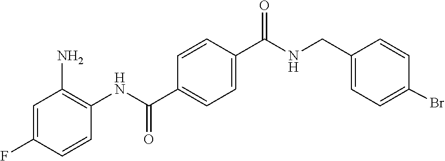 [SMILES: Nc1cc(F)ccc1NC(=O)c1ccc(C(=O)NCc2ccc(Br)cc2)cc1]

Compound DL-HDAC13. 1.1 mg, yield: 94%; yellow powder. MS (ESI) calcd. for C₂₁H₁₇BrFN₃O₂: 442.29, Found: 441.86, [M+1] 443.80, 444.37, 445.09.

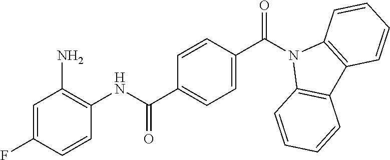 [SMILES: Nc1cc(F)ccc1NC(=O)c1ccc(C(=O)n2c3ccccc3c3ccccc32)cc1]

Compound DL-HDAC14. 1.2 mg, yield: 89%; yellow powder. MS (ESI) calcd. for C₂₆H₁₈FN₃O₂: 423.45, Found: [M+1] 423.92.

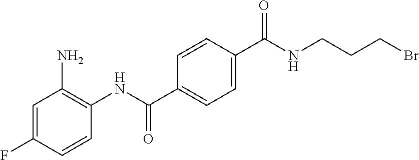 [SMILES: Nc1cc(F)ccc1NC(=O)c1ccc(C(=O)NCCCBr)cc1]

Compound DL-HDAC15. 3.9 mg, yield: 92%; white powder. ¹H NMR (500 MHz, DMSO-d₆) δ 9.70 (s, 1H), 8.69 (t, J=5.6 Hz, 1H), 8.09-8.01 (m, 2H), 7.97-7.91 (m, 2H), 7.12 (ddd, J=8.9, 6.4, 2.3 Hz, 1H), 6.55 (dd, J=11.2, 2.9 Hz, 1H), 6.37 (td, J=8.5, 2.9 Hz, 1H), 5.26 (s, 1H), 3.60 (t, J=6.6 Hz, 2H), 3.43 (s, 2H), 2.10 (p, J=6.7 Hz, 2H). MS (ESI) calcd. for C₁₇H₁₇BrFN₃O₂: 394.24, Found: 393.86, [M+1] 395.80, 396.41, 397.21.

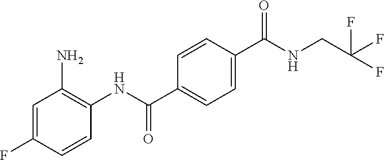 [SMILES: Nc1cc(F)ccc1NC(=O)c1ccc(C(=O)NCC(F)(F)F)cc1]

Compound DL-HDAC16. 1.1 mg, yield: 86%; yellow powder. MS (ESI) calcd. for C₁₈H₁₃F₄N₃O₂: 355.29, Found: 355.74, [M+1] 356.62, 357.23.

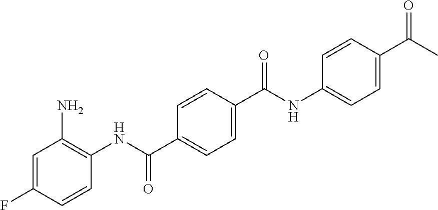 [SMILES: CC(=O)c1ccc(NC(=O)c2ccc(C(=O)Nc3ccc(F)cc3N)cc2)cc1]

Compound DL-HDAC17. 4.4 mg, yield: 88%; white powder. ¹H NMR (500 MHz, DMSO-d₆) δ 10.69 (s, 1H), 9.75 (d, J=12.4 Hz, 1H), 8.13-7.97 (m, 8H), 7.16-7.11 (m, 1H), 6.57-6.53 (m, 1H), 6.40-6.34 (m, 1H), 2.57 (s, 3H). MS (ESI) calcd. for C₂₂H₁₈FN₃O₃: 391.40, Found: [M+1] 392.19.

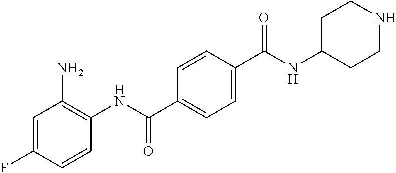 [SMILES: Nc1cc(F)ccc1NC(=O)c1ccc(C(=O)NC2CCNCC2)cc1]

Compound DL-HDAC18. 3.4 mg, yield: 87%; yellow powder. ¹H NMR (500 MHz, DMSO-d₆) δ 9.71 (s, 1H), 8.34 (s, 1H), 8.06 (dd, J=8.3, 2.0 Hz, 2H), 7.97-7.93 (m, 2H), 7.23 (s, 1H), 7.12 (dd, J=8.7, 6.3 Hz, 1H), 6.90 (s, 1H), 6.55 (dd, J=11.2, 2.9 Hz, 1H), 6.37 (td, J=8.5, 2.9 Hz, 1H), 5.26 (s, 2H), 4.04 (t, J=6.9 Hz, 2H), 3.27 (q, J=6.6 Hz, 2H), 1.99 (p, J=6.9 Hz, 2H). MS (ESI) calcd. for C₁₉H₂₁FN₄O₂: 356.40, Found: [M+1] 357.23.

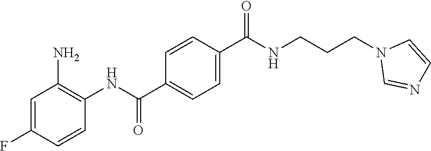 [SMILES: Nc1cc(F)ccc1NC(=O)c1ccc(C(=O)NCCCn2ccnc2)cc1]

Compound DL-HDAC19. 6.7 mg, yield: 91%; yellow powder. ¹H NMR (500 MHz, DMSO-d₆) δ 9.75 (s, 1H), 8.57 (d, J=7.5 Hz, 1H), 8.39 (s, 1H), 8.06 (d, J=8.1 Hz, 2H), 7.97 (d, J=8.1 Hz, 2H), 7.17-7.05 (m, 1H), 6.55 (dd, J=11.2, 3.0 Hz, 1H), 6.37 (td, J=8.6, 2.9 Hz, 1H), 5.26 (s, 2H), 2.86 (t, J=12.2 Hz, 2H), 1.92 (d, J=12.8 Hz, 2H), 1.68 (d, J=12.0 Hz, 2H). MS (ESI) calcd. for C₂₀H₂₀FN₅O₂: 381.41, Found: [M+1] 382.27.

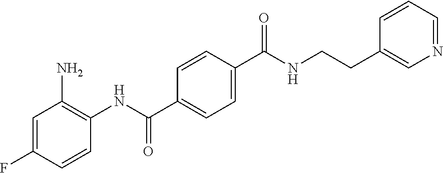 [SMILES: Nc1cc(F)ccc1NC(=O)c1ccc(C(=O)NCCc2cccnc2)cc1]

Compound DL-HDAC20. 5.8 mg, yield: 93%; white powder. ¹H NMR (500 MHz, DMSO-d₆) δ 9.62 (s, 1H), 8.65 (t, J=5.5 Hz, 1H), 8.35 (dd, J=4.8, 1.7 Hz, 1H), 7.97 (d, J=8.6 Hz, 2H), 7.86-7.81 (m, 2H), 7.62-7.59 (m, 1H), 7.25 (ddd, J=7.8, 4.8, 0.9 Hz, 1H), 7.05 (dd, J=8.7, 6.3 Hz, 1H), 6.48 (dt, J=11.3, 2.5 Hz, 1H), 6.29 (td, J=8.5, 2.8 Hz, 1H), 5.19 (s, 2H), 3.47 (dd, J=7.3, 5.8 Hz, 2H), 2.83 (t, J=7.1 Hz, 2H). MS (ESI) calcd. for C₂₁H₁₉FN₄O₂: 378.41, Found: [M+1] 379.19, 382.23.

### Example 2—Synthesis of Biotin-LW3

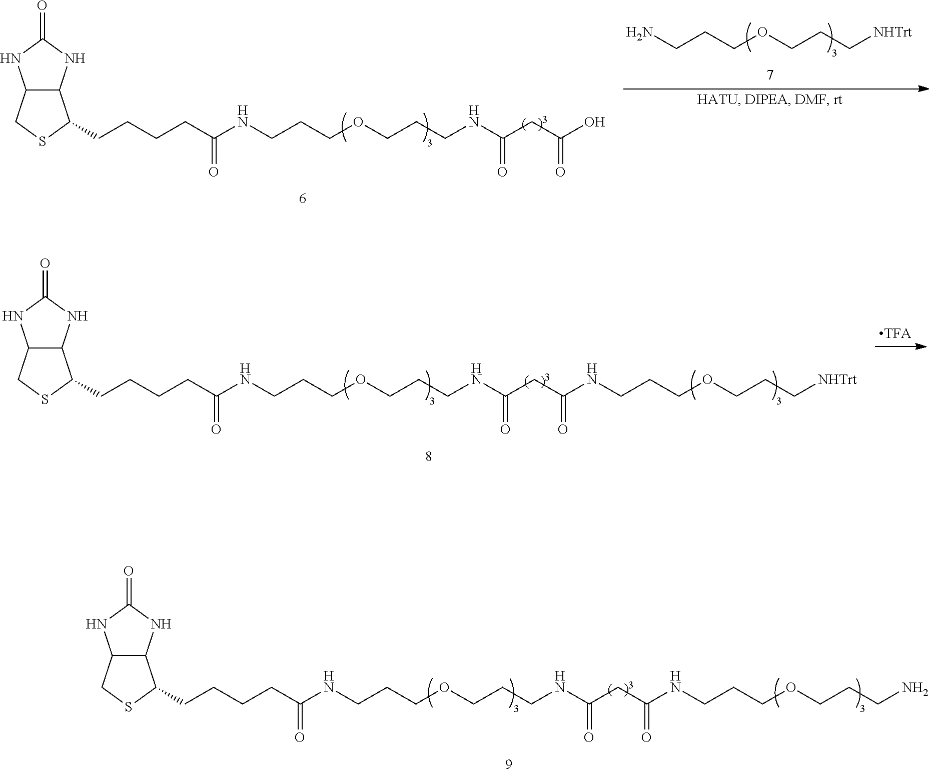

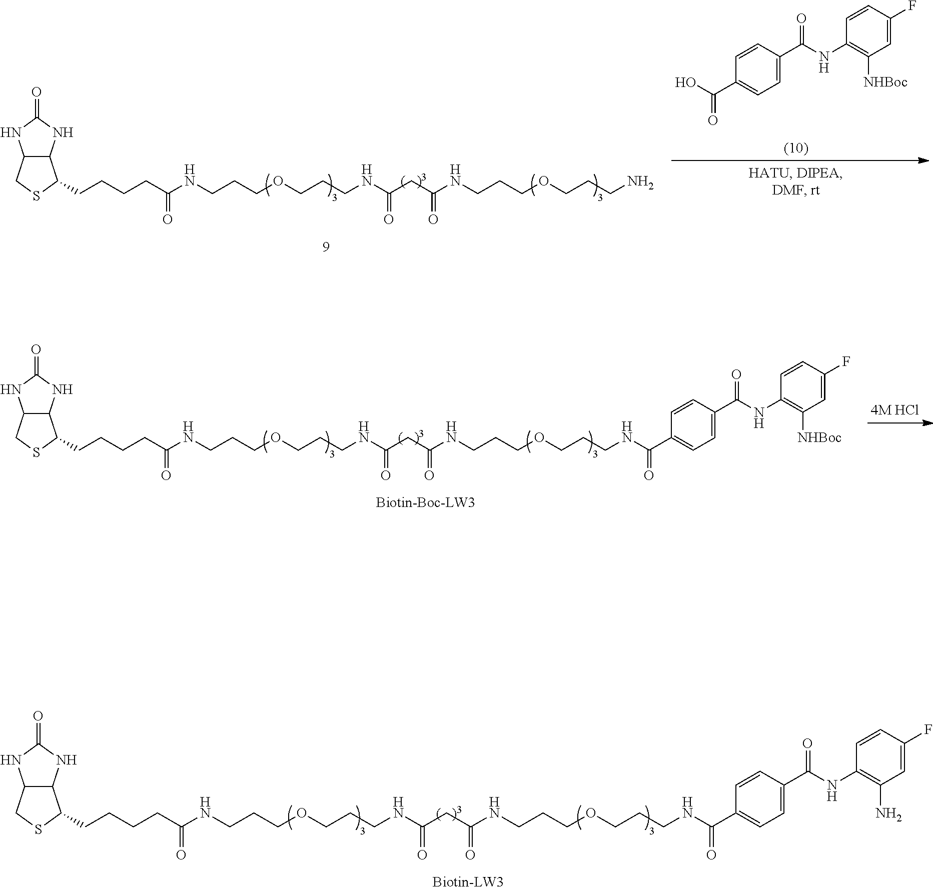

Step 1:

In a reaction flask, to the mixture of compound 6 (70 mg, 1 eq.), compound 7 (56.4 mg, 1.2 eq.) and HATU (42.4 mg, 1.1 eq.) was added DMF (0.4 mL) as the solvent. Then DIPEA (33.9 uL, 2 eq.) was added as the base. The reaction mixture was stirred at room temperature for 0.5 h. After the reaction was complete, the reaction mixture was purified directly via Pre-HPLC (MeCN/water, 0.1% formic acid) to give compound 8 (71 mg) as yellow powder after lyophilization. Yield: 70%. MS (ESI) calcd. for C₅₄H₈₀N₆O₁₀S: 1004.57, Found: [M+1] 1005.92, 1006.90. ¹H NMR (500 MHz, Chloroform-d) δ 9.86 (s, 2H), 7.77 (s, 2H), 7.55-7.37 (m, 15H), 4.63 (dd, J=7.9, 4.7 Hz, 1H), 4.44 (dd, J=8.0, 4.5 Hz, 1H), 3.80-3.53 (m, 18H), 3.53-3.43 (m, 6H), 3.37 (dt, J=17.2, 6.1 Hz, 6H), 3.27-3.11 (m, 3H), 2.97 (dd, J=13.1, 4.9 Hz, 1H), 2.81 (d, J=13.1 Hz, 1H), 2.35 (dt, J=19.6, 5.3 Hz, 6H), 2.10-2.02 (m, 2H), 1.90 (s, 2H), 1.84-1.65 (m, 9H), 1.59-1.40 (m, 2H).

Step 2:

In a reaction flask, to the mixture of compound 8 (50 mg) dissolved in dichloromethane (4 mL) was added TFA (1 mL). The reaction mixture was stirred at room temperature for 3 h. After the reaction was complete, the reaction mixture was concentrated under reduced pressure to remove the solvent and TFA, the obtained residue compound 9 was used for the next step reaction directly. Yield: ˜100%

Step 3:

In a reaction flask, to the mixture of compound 9 (20 mg, 1 eq.), compound 10 (1.2 eq.) and HATU (22.9 mg, 1.1 eq.) was added DMF (0.3 mL) as the solvent. Then DIPEA (44.6 uL, 5 eq.) was added as the base. The reaction mixture was stirred at room temperature for 0.25 h. After the reaction was complete, the reaction mixture was purified directly via Pre-HPLC (MeCN/Water, 0.1% formic acid, twice) to give intermediate Biolin-Boc-LW3 20 mg as yellow powder after lyophilization. Yield: 33%. MS (ESI) calcd. for C₅₄H₈₃FN₈O₁₄S: 1118.57, Found: [M+1] 1120.00, 1120.95.

Step 4:

In a reaction flask, to the compound Biolin-Boc-LW3 (20 mg) was added 4M HCl/dioxane (2 mL). The reaction mixture was stirred at room temperature for 4 h. After the reaction was complete, the reaction mixture was diluted in 5 mL water and subjected to lyophilization to produce Biolin-LW3. 18 mg, Yield: ˜100%. MS (ESI) calcd. for C₄₉H₇₅FN₈O₁₂S: 1018.52, Found: [M+1] 1019.86, 1020.77. ¹H NMR (500 MHz, Methanol-d₄) δ 8.17 (d, J=8.0 Hz, 2H), 8.01 (d, J=8.1 Hz, 2H), 7.57 (dd, J=9.6, 5.3 Hz, 1H), 7.37 (td, J=8.2, 7.3, 2.7 Hz, 2H), 4.60 (s, 1H), 4.41 (s, 1H), 3.76 (dd, J=6.2, 4.9 Hz, 3H), 3.70-3.66 (m, 10H), 3.60 (dq, J=6.0, 3.8, 3.4 Hz, 10H), 3.53 (dd, J=6.9, 3.2 Hz, 7H), 3.31-3.26 (m, 6H), 2.98 (dd, J=12.9, 4.3 Hz, 1H), 2.77 (d, J=12.7 Hz, 1H), 2.26 (q, J=7.3 Hz, 6H), 1.91 (dt, J=17.9, 6.7 Hz, 4H), 1.77 (h, J=6.3 Hz, 7H), 1.46 (p, J=7.5 Hz, 2H).

### Example 3—HDAC Enzyme Assays

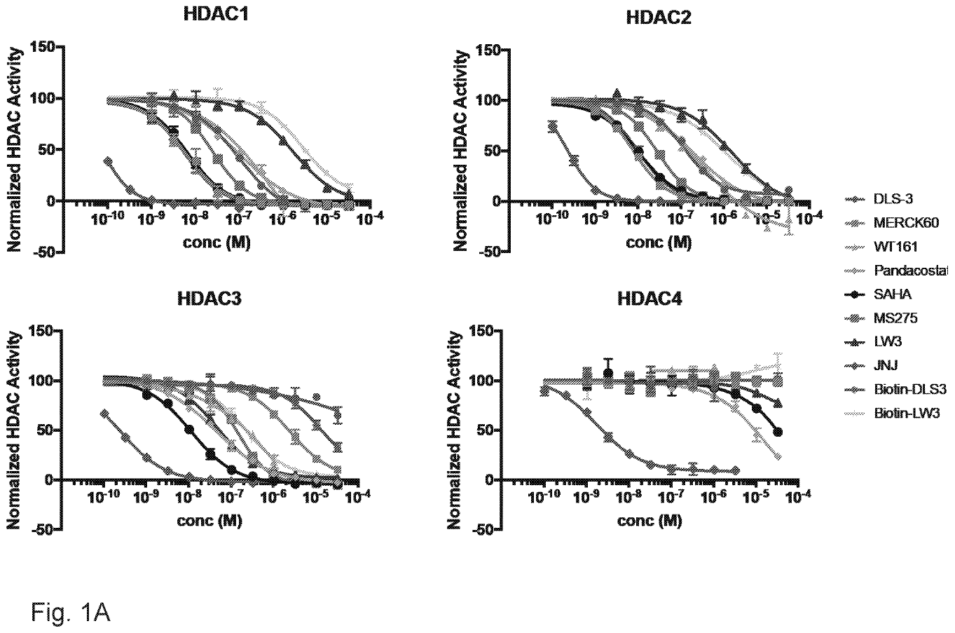

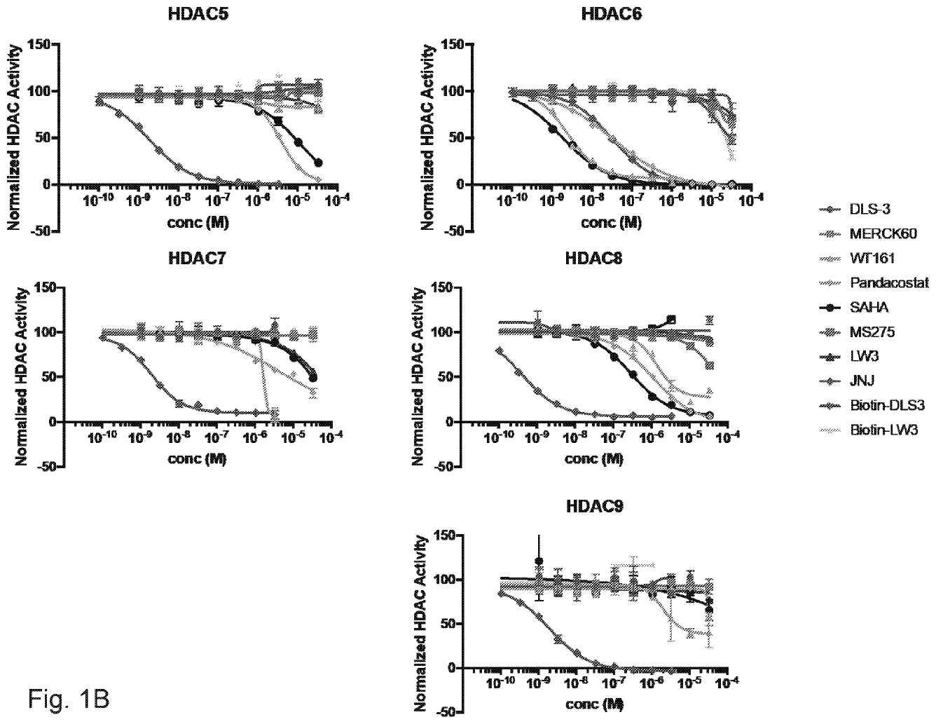

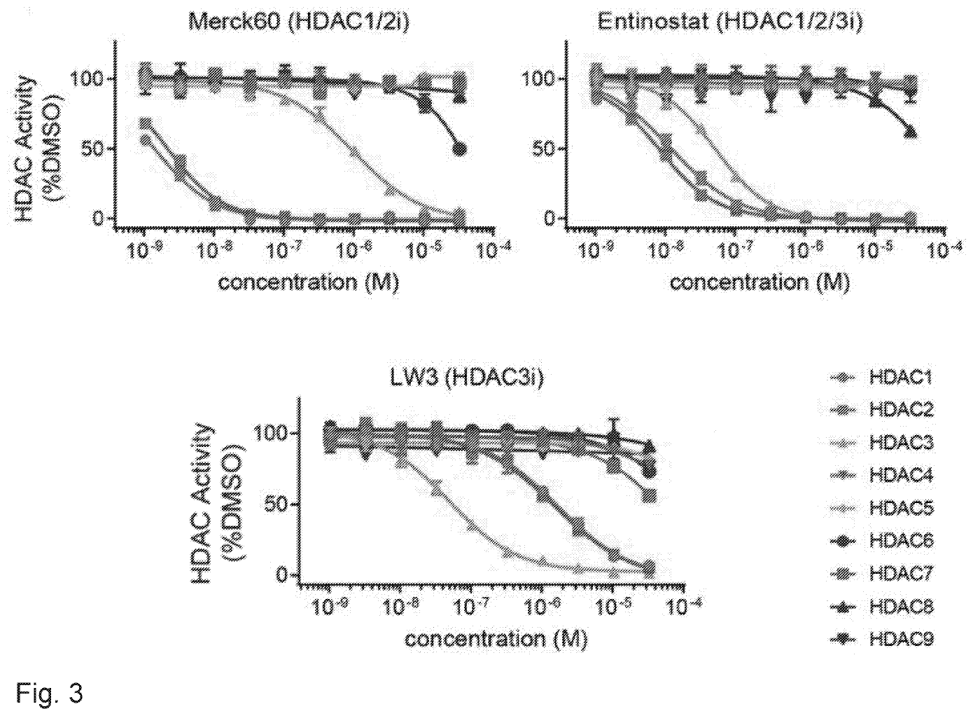

The results of HDAC inhibition assays can be found in FIGS. 1A, 1B, 2, and 3. As can be seen, a compound of Formula I (LW3) selectively inhibits HDAC3 over other HDAC isoforms.

The inhibitory effect of compounds on HDAC1-HDAC9 function was determined in vitro using an optimized homogenous assay performed in a 384-well plate format. In this assay, recombinant, full-length HDAC protein (HDAC1 100 pg/ul, HDAC2 200 pg/ul, HDAC3 100 pg/ul, HDAC4 0.5 pg/ul, HDAC5 10 pg/ul, HDAC6 350 pg/ul, HDAC7 2 pg/ul, HDAC8 16 pg/ul, HDAC9 20 pg/ul; BPS Biosciences, San Diego, Calif., USA) was incubated with inhibitory compound for 3 hours, and then fluorophore-conjugated substrates MAZ1600 and MAZ1675 were added at a concentration equivalent to the substrate Km (MAZ1600: 8.9 μM for HDAC1, 10.5 μM for HDAC2, 7.9 μM for HDAC3, and 9.4 μM for HDAC6. MAZ1675: 11.5 μM for HDAC4, 64.7 μM for HDAC5, 29.6 μM for HDAC7, 202.2 μM for HDAC8 and 44.3 μM for HDAC9). Reactions were performed in assay buffer (50 mM HEPES, 100 mM KCl, 0.001% (v/v) Tween 20, 0.05% (w/v) bovine serum albumin, 200 μM TCEP, pH 7.4) and followed for fluorogenic release of 7-amino-4-methylcoumarin from substrate upon deacetylase and trypsin enzymatic activity. Trypsin was present at a final concentration of 50 nM (Worthington Biochemical Corporation). Fluorescence measurements were obtained approximately every 5 min using a multilabel plate reader and plate stacker (Envision, Perkin-Elmer). Data were analyzed on a plate-by-plate basis for the linear range of fluorescence overtime. The first derivative of data obtained from the plate capture corresponding to the mid-linear range was imported into analytical software (Spotfire DecisionSite and GraphPad Prism). Replicate experimental data from incubations with inhibitor were normalized to DMSO controls. IC₅₀ is determined by logistic regression with unconstrained maximum and minimum values.

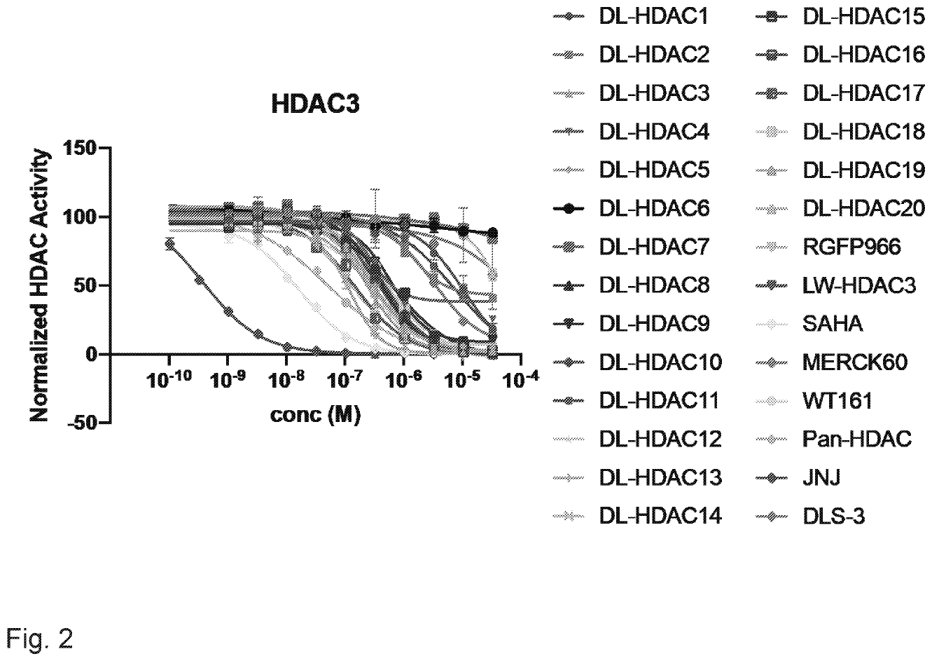

Table 3 summarizes the data of FIG. 2 and shows the IC₅₀ data for various compounds against HDAC3.

TABLE 3

| Compounds | IC₅₀ (uM) |
|---|---|
| DL-HDAC1 | 10.49 |
| DL-HDAC2 | 1.33 |
| DL-HDAC3 | 0.23 |
| DL-HDAC4 | 0.27 |
| DL-HDAC5 | 0.26 |
| DL-HDAC6 | N/A |
| DL-HDAC7 | N/A |
| DL-HDAC8 | 0.41 |
| DL-HDAC9 | 0.63 |
| DL-HDAC10 | 0.45 |
| DL-HDAC11 | 7.77 |
| DL-HDAC12 | 0.27 |
| DL-HDAC13 | 0.12 |
| DL-HDAC14 | N/A |
| DL-HDAC15 | 0.71 |
| DL-HDAC16 | 0.39 |
| DL-HDAC17 | 0.15 |
| DL-HDAC18 | 0.53 |
| DL-HDAC19 | 0.27 |
| DL-HDAC20 | 0.29 |
| RGFP966 | 0.12 |
| LW-HDAC3 | 0.39 |
| SAHA | 0.016 |
| MERCK60 | 3.28 |
| WT161 | 4.04 |
| Pan-HDAC | 0.052 |
| JNJ | 0.00038 |
| DLS-3 | N/A |

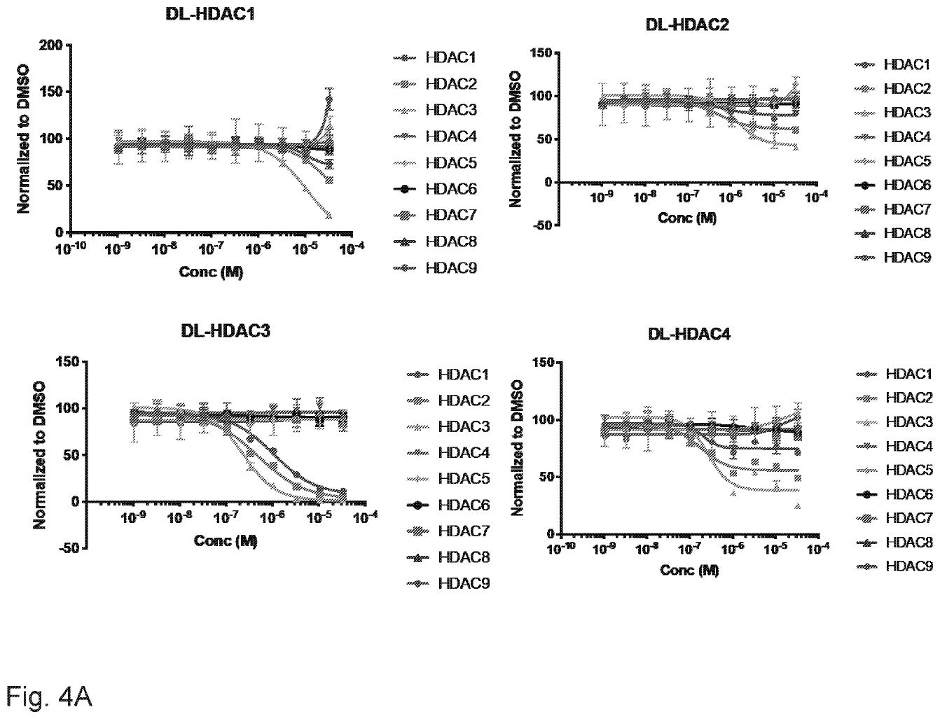

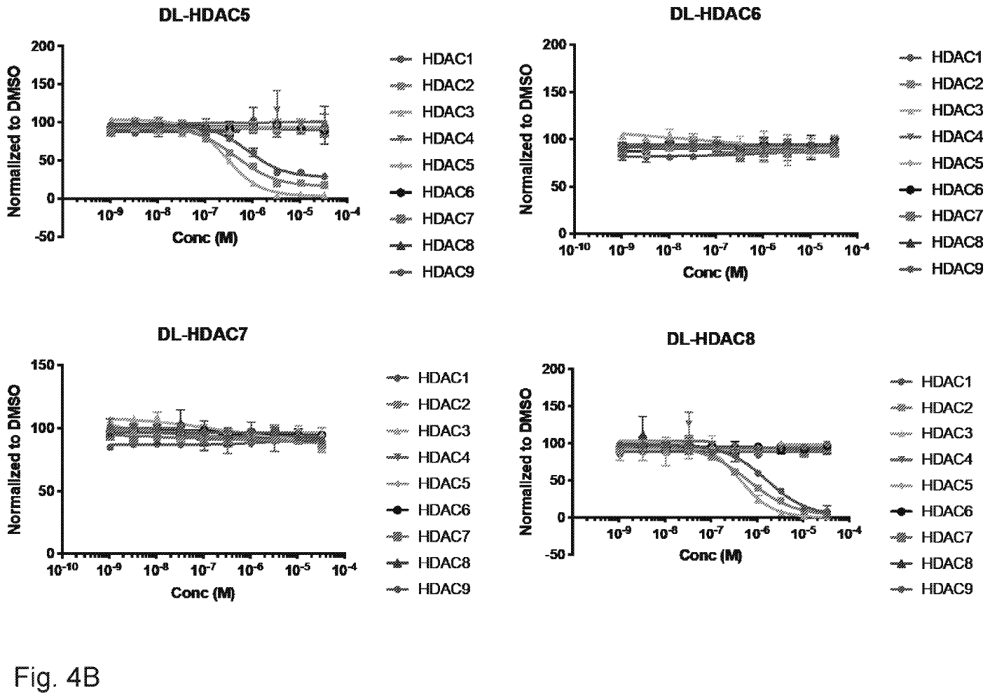

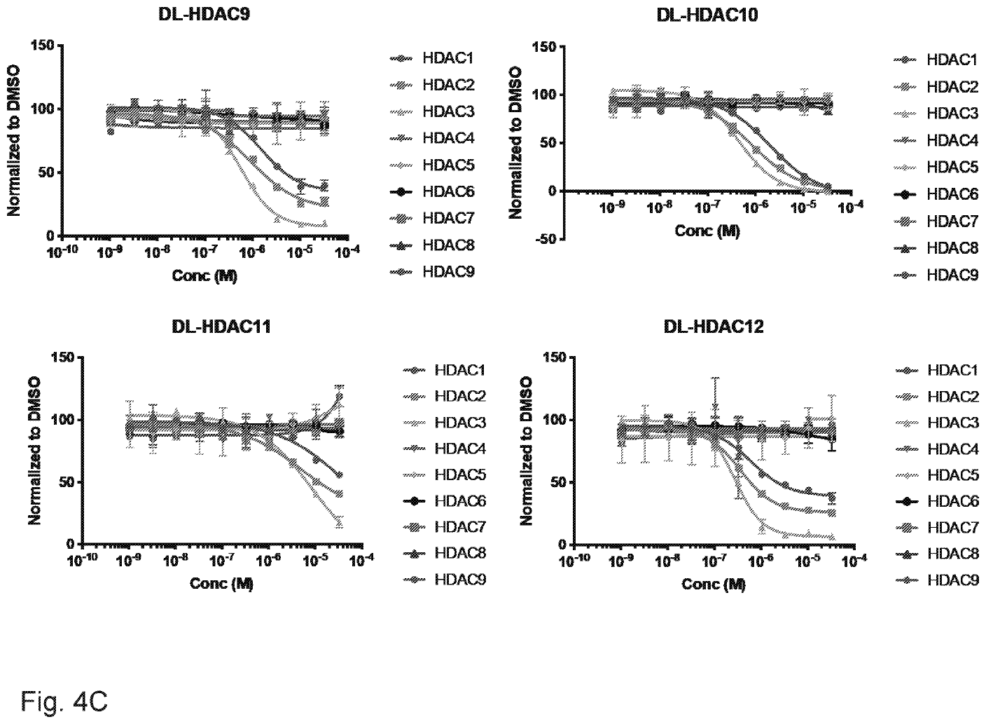

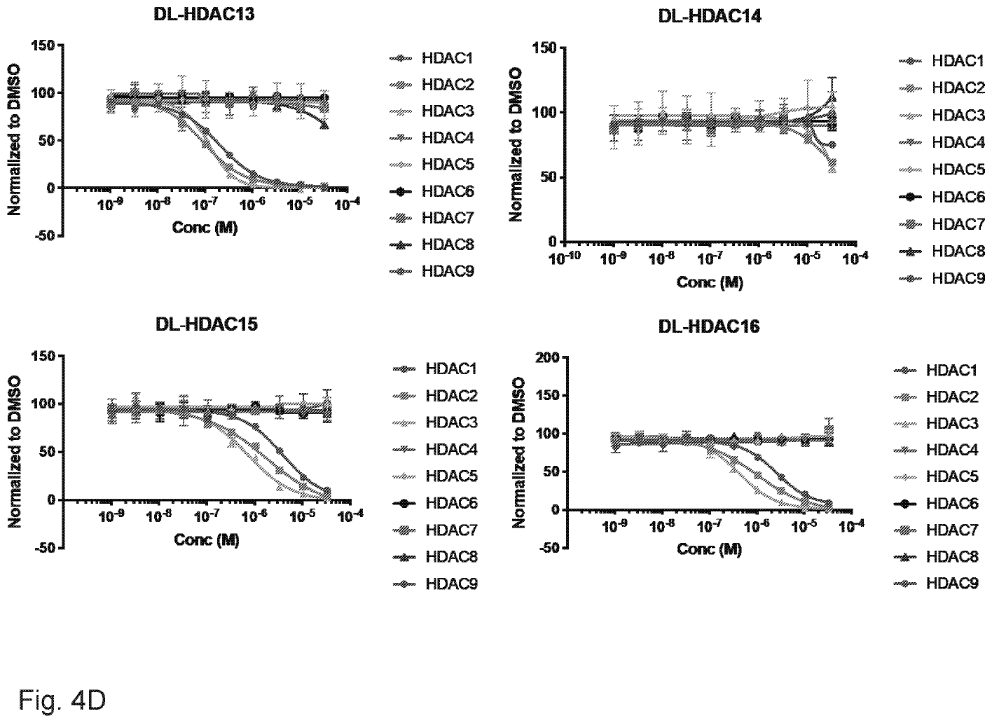

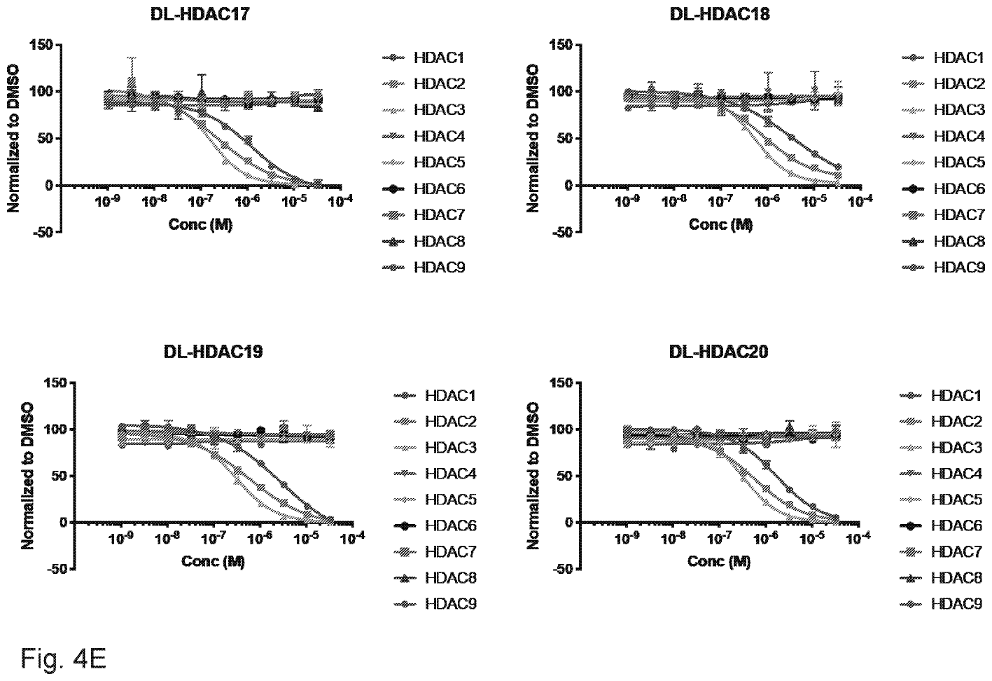

The inhibitory activity of the compounds disclosed herein were tested for all HDAC isoforms to compare the selectivity for HDAC3 against HDACS 1, 2, and 4-9. The results of these studies can be found in FIGS. 4A-4D. The IC₅₀ values for the compounds of Formula I are also summarized in Tables 4-23 below.

TABLE 4
DL-HDAC1

| | IC50 (uM) |
|---|---|
| HDAC1 | N/A |
| HDAC2 | N/A |
| HDAC3 | 11.07 |
| HDAC4 | N/A |
| HDAC5 | N/A |
| HDAC6 | N/A |
| HDAC7 | N/A |
| HDAC8 | N/A |
| HDAC9 | N/A |

TABLE 5
DL-HDAC2

| | IC50 (uM) |
|---|---|
| HDAC1 | N/A |
| HDAC2 | N/A |
| HDAC3 | 1.33 |
| HDAC4 | N/A |
| HDAC5 | N/A |
| HDAC6 | N/A |
| HDAC7 | N/A |
| HDAC8 | N/A |
| HDAC9 | N/A |

TABLE 6
DL-HDAC3

| | IC50 (uM) |
|---|---|
| HDAC1 | 1.05 |
| HDAC2 | 0.49 |
| HDAC3 | 0.23 |
| HDAC4 | N/A |
| HDAC5 | N/A |
| HDAC6 | N/A |
| HDAC7 | N/A |
| HDAC8 | N/A |
| HDAC9 | N/A |

TABLE 7
DL-HDAC4

| | IC50 (uM) |
|---|---|
| HDAC1 | N/A |
| HDAC2 | N/A |
| HDAC3 | 0.26 |
| HDAC4 | N/A |
| HDAC5 | N/A |
| HDAC6 | N/A |
| HDAC7 | N/A |
| HDAC8 | N/A |
| HDAC9 | N/A |

TABLE 8
DL-HDAC5

| | IC50 (uM) |
|---|---|
| HDAC1 | 0.81 |
| HDAC2 | 0.44 |
| HDAC3 | 0.28 |
| HDAC4 | N/A |
| HDAC5 | N/A |
| HDAC6 | N/A |
| HDAC7 | N/A |
| HDAC8 | N/A |
| HDAC9 | N/A |

TABLE 9
DL-HDAC6

| | IC50 (uM) |
|---|---|
| HDAC1 | N/A |
| HDAC2 | N/A |
| HDAC3 | N/A |
| HDAC4 | N/A |
| HDAC5 | N/A |
| HDAC6 | N/A |
| HDAC7 | N/A |
| HDAC8 | N/A |
| HDAC9 | N/A |

TABLE 10
DL-HDAC7

| | IC50 (uM) |
|---|---|
| HDAC1 | N/A |
| HDAC2 | N/A |
| HDAC3 | N/A |
| HDAC4 | N/A |
| HDAC5 | N/A |
| HDAC6 | N/A |
| HDAC7 | N/A |
| HDAC8 | N/A |
| HDAC9 | N/A |

TABLE 11
DL-HDAC8

| | IC50 (uM) |
|---|---|
| HDAC1 | 1.65 |
| HDAC2 | 0.66 |
| HDAC3 | 0.42 |
| HDAC4 | N/A |
| HDAC5 | N/A |
| HDAC6 | N/A |
| HDAC7 | N/A |
| HDAC8 | N/A |
| HDAC9 | N/A |

TABLE 12
DL-HDAC9

| | IC50 (uM) |
|---|---|
| HDAC1 | 1.55 |
| HDAC2 | 0.93 |
| HDAC3 | 0.55 |
| HDAC4 | N/A |
| HDAC5 | N/A |
| HDAC6 | N/A |
| HDAC7 | N/A |
| HDAC8 | N/A |
| HDAC9 | N/A |

TABLE 13
DL-HDAC10

| | IC50 (uM) |
|---|---|
| HDAC1 | 2.07 |
| HDAC2 | 0.85 |
| HDAC3 | 0.45 |
| HDAC4 | N/A |
| HDAC5 | N/A |
| HDAC6 | N/A |
| HDAC7 | N/A |
| HDAC8 | N/A |
| HDAC9 | N/A |

TABLE 14
DL-HDAC11

| | IC50 (uM) |
|---|---|
| HDAC1 | 36.01 |
| HDAC2 | 4.69 |
| HDAC3 | 8.81 |
| HDAC4 | N/A |
| HDAC5 | N/A |
| HDAC6 | N/A |
| HDAC7 | N/A |
| HDAC8 | N/A |
| HDAC9 | N/A |

TABLE 15
DL-HDAC12

| | IC50 (uM) |
|---|---|
| HDAC1 | 0.6 |
| HDAC2 | 0.36 |
| HDAC3 | 0.27 |
| HDAC4 | N/A |
| HDAC5 | N/A |
| HDAC6 | N/A |
| HDAC7 | N/A |
| HDAC8 | N/A |
| HDAC9 | N/A |

TABLE 16
DL-HDAC13

| | IC50 (uM) |
|---|---|
| HDAC1 | 0.2 |
| HDAC2 | 0.094 |
| HDAC3 | 0.12 |
| HDAC4 | N/A |
| HDAC5 | N/A |
| HDAC6 | N/A |
| HDAC7 | N/A |
| HDAC8 | N/A |
| HDAC9 | N/A |

TABLE 17
DL-HDAC14

| | IC50 (uM) |
|---|---|
| HDAC1 | N/A |
| HDAC2 | N/A |
| HDAC3 | N/A |
| HDAC4 | N/A |
| HDAC5 | N/A |
| HDAC6 | N/A |
| HDAC7 | N/A |
| HDAC8 | N/A |
| HDAC9 | N/A |

TABLE 18
DL-HDAC15

| | IC50 (uM) |
|---|---|
| HDAC1 | 3.92 |
| HDAC2 | 1.8 |
| HDAC3 | 0.73 |
| HDAC4 | N/A |
| HDAC5 | N/A |
| HDAC6 | N/A |
| HDAC7 | N/A |
| HDAC8 | N/A |
| HDAC9 | N/A |

TABLE 19
DL-HDAC16

| | IC50 (uM) |
|---|---|
| HDAC1 | 2.62 |
| HDAC2 | 0.99 |
| HDAC3 | 0.42 |
| HDAC4 | N/A |
| HDAC5 | N/A |
| HDAC6 | N/A |
| HDAC7 | N/A |
| HDAC8 | N/A |
| HDAC9 | N/A |

TABLE 20
DL-HDAC17

| | IC50 (uM) |
|---|---|
| HDAC1 | 1.22 |
| HDAC2 | 0.22 |
| HDAC3 | 0.17 |
| HDAC4 | N/A |
| HDAC5 | N/A |
| HDAC6 | N/A |
| HDAC7 | N/A |
| HDAC8 | N/A |
| HDAC9 | N/A |

TABLE 21
DL-HDAC18

| | IC50 (uM) |
|---|---|
| HDAC1 | 3.49 |
| HDAC2 | 0.88 |
| HDAC3 | 0.56 |
| HDAC4 | N/A |
| HDAC5 | N/A |
| HDAC6 | N/A |
| HDAC7 | N/A |
| HDAC8 | N/A |
| HDAC9 | N/A |

TABLE 22
DL-HDAC19

| | IC50 (uM) |
|---|---|
| HDAC1 | 2.79 |
| HDAC2 | 0.61 |
| HDAC3 | 0.29 |
| HDAC4 | N/A |
| HDAC5 | N/A |
| HDAC6 | N/A |
| HDAC7 | N/A |
| HDAC8 | N/A |
| HDAC9 | N/A |

TABLE 23
DL-HDAC20

| | IC50 (uM) |
|---|---|
| HDAC1 | 1.73 |
| HDAC2 | 0.59 |
| HDAC3 | 0.33 |
| HDAC4 | N/A |
| HDAC5 | N/A |
| HDAC6 | N/A |
| HDAC7 | N/A |
| HDAC8 | N/A |
| HDAC9 | N/A |

The disclosed subject matter is not to be limited in scope by the specific embodiments and examples described herein. Indeed, various modifications of the disclosure in addition to those described will become apparent to those skilled in the art from the foregoing description and accompanying figures. Such modifications are intended to fall within the scope of the appended claims.

All references (e.g., publications or patents or patent applications) cited herein are incorporated herein by reference in their entirety and for all purposes to the same extent as if each individual reference (e.g., publication or patent or patent application) was specifically and individually indicated to be incorporated by reference in its entirety for all purposes.

Other embodiments are within the following claims.

1. A compound of Formula I: 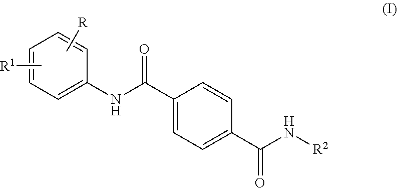
or a pharmaceutically acceptable salt thereof;
wherein:
R is selected from the group consisting of fluoro, bromo, chloro, —NH₂, —OH, —SH, —NHR³, —N(R³)₂, OR³, SR³, NO₂, thienyl, and CN;
R¹ is selected from the group consisting of fluoro, bromo, chloro, —NH₂, —OH, —SH, —NHR³, —N(R³)₂, OR³, SR³, NO₂, thienyl, and CN;
R² is selected from the group consisting of C₆-C₁₀ aryl, C₅-C₁₃ heteroaryl, C₃-C₁₀ cycloalkyl, C₃-C₁₀ heterocycloalkyl, C₁-C₆ alkyl-C₆-C₁₀ aryl, C₁-C₆ alkyl-C₅-C₁₃ heteroaryl, C₁-C₆ alkyl-C₃-C₁₀ cycloalkyl, C₁-C₆ alkyl-C₃-C₁₀ heterocycloalkyl, and -linker-biotin;
wherein C₅-C₁₀ heteroaryl, C₆-C₁₀ aryl, and C₁-C₆ alkyl are optionally substituted with one to three halo, phenyl, —C(O)Me, —OMe, methyl, NO₂, —SO₂Me, Ce heterocycloalkyl, C₅-C₆ heteroaryl, and CF₃; and
R³ is independently, at each occurrence, selected from the group consisting of H, C₁-C₆ alkyl, and C₁-C₆ alkoxy.

2. The compound of claim 1 or a pharmaceutically acceptable salt thereof, wherein
R is selected from the group consisting of fluoro, bromo, chloro, —NH₂, —OH, and —SH;
R¹ is selected from the group consisting of fluoro, bromo, chloro, —NH₂, —OH, and —SH;
R² is C₆-C₁₀ aryl or C₅-C₁₃ heteroaryl; and
R³ is H or C₁-C₆ alkyl.

3. The compound of claim 1 or 2, or a pharmaceutically acceptable salt thereof, wherein R is fluoro and R¹ is —NH₂.

4. The compound of any of claims 1-3, or a pharmaceutically acceptable salt thereof, wherein R² is —CH₂C₅-C₁₃ heteroaryl.

5. The compound of claim 1, wherein the compound of Formula I is a compound of Formula II: 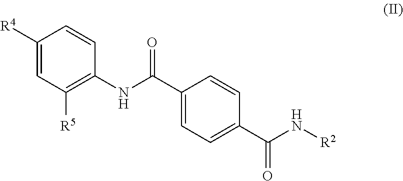
or a pharmaceutically acceptable salt thereof;
wherein:
R² is selected from the group consisting of C₆-C₁₀ aryl, C₅-C₁₀ heteroaryl, or linker-biotin, wherein C₆-C₁₀ aryl is optionally substituted with halo or SO₂Me;
R⁴ is selected from the group consisting of fluoro, bromo, chloro, —NH₂, —OH, and —SH; and
R⁵ is selected from the group consisting of fluoro, bromo, chloro, —NH₂, —OH, and —SH.

6. The compound of claim 5, or a pharmaceutically acceptable salt thereof, wherein R² is C₅-C₁₀ heteroaryl, R⁴ is fluoro, and R⁵ is —NH₂.

7. The compound of claim 5, wherein R₂ is

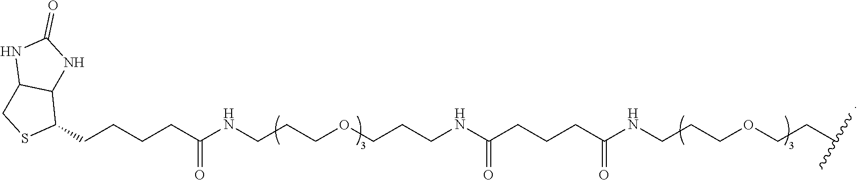

8. The compound of any of claims 1-6, or a pharmaceutically acceptable salt thereof, wherein the compound of Formula I or Formula II is a compound of Formula III:

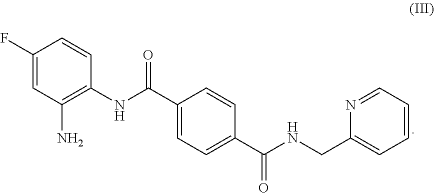 [SMILES: Nc1cc(F)ccc1NC(=O)c1ccc(C(=O)NCc2ccccn2)cc1]

9. The compound of claim 6, wherein the compound of Formula II is a compound of Formula IV: 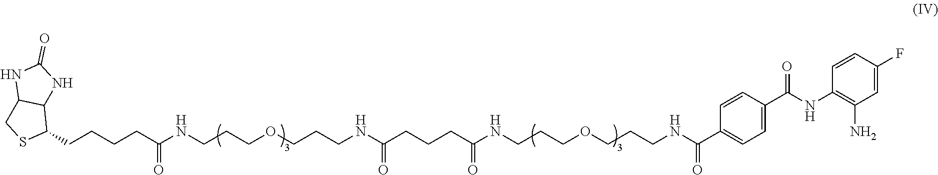
or a pharmaceutically acceptable salt thereof.

10. A pharmaceutical composition comprising a compound according to any one of claims 1-9, or a salt thereof, and at least one pharmaceutically acceptable carrier.

11. A method of selectively inhibiting the activity of histone deacetylase 3 (HDAC3) in an individual in need thereof, comprising administering to the individual a therapeutically effective amount of a compound according to any one of claims 1-9 or a composition according to claim 10.

12. A method of treating cancer in an individual in need thereof, comprising administering to the individual a therapeutically effective amount of a compound according to any one of claims 1-9 or a composition according to claim 10.

13. The method of claim 12, wherein the cancer is Medulloblastoma, rhabdomyosarcoma, Hodgkin lymphoma, acute myeloid leukemia, myelodysplastic syndrome, pancreatic cancer, colon cancer, ovarian cancer, lung cancer, stomach cancer, a muscle cancer, a bone cancer, or a skin cancer.

14. The method of claim 13, wherein the cancer is rhabdomyosarcoma.

15. The method of claim 13, wherein the cancer is alveloar rhabdomyosarcoma

16. The method of claim 13, wherein the cancer is pediatric rhabdomyosarcoma.

17. A method of treating a neurodegenerative disease in an individual in need thereof, comprising administering to the individual a therapeutically effective amount of a compound according to any one of claims 1-9 or a composition according to claim 10.

18. The method of claim 17, wherein the neurodegenerative disease is Spinal Muscular Atrophy, polyglutamine-related diseases, or amyotrophic lateral sclerosis.

19. The method of claim 18, wherein the polyglutamine-related disease is Huntington disease, dentatorubral-pallidoluysian atrophy, or spinocerebellar ataxia type 6 (SCA6).

20. A process for preparing a compound of Formula V: 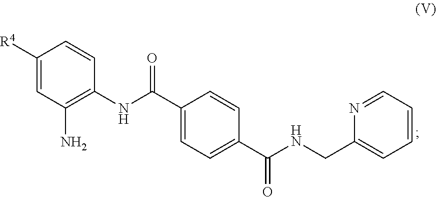
comprising reacting a corn und of Formula VI: 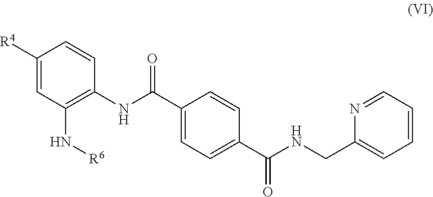
with an acid in a solvent;
wherein
R⁴ is selected from the group consisting of fluoro, bromo, and chloro; and
R⁶ is a protecting group selected from the group consisting of acetyl (Ac), benzyl (Bn), tert-butyloxycarbonyl (Boc), benzoyl (Bz), carboxybenzyl (Cbz), carbamate, 3,4-dimethoxy-benzyl (DMPM), 9-fluorenylmethyloxycarbonyl (Fmoc), p-methoxybenzyl carbonyl (Moz), 4-nitrobenzylsulfonyl (Nos), p-methoxybenzyl (PMB), p-methoxyphenyl (PMP), 4-toluenesulfonyl (Tos), and trichloroethyl chloroformate (Troc).

21. The process of claim 20, wherein the acid is hydrochloric acid.

22. The process of claim 20 or 21, wherein the solvent is dioxane.

23. The process of any one of claims 20-22, wherein R⁶ is tert-butyloxycarbonyl (Boc).

24. The process of any one of claims 20-23, wherein R⁴ is fluoro.

25. A process for preparing a compound of Formula VI: 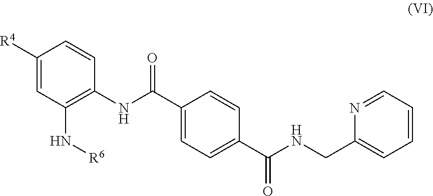
comprising reacting a compound of Formula VII: 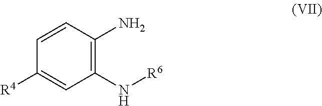
with a compound of Formula VIII: 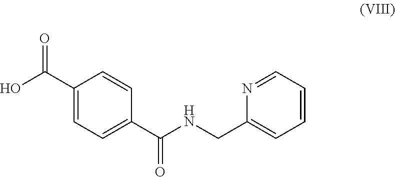 [SMILES: O=C(O)c1ccc(C(=O)NCc2ccccn2)cc1]
in the presence of a peptide coupling reagent, a base, and a solvent;
wherein
R⁴ is selected from the group consisting of fluoro, bromo, and chloro; and
R⁶ is a protecting group selected from the group consisting of acetyl (Ac), benzyl (Bn), tert-butyloxycarbonyl (Boc), benzoyl (Bz), carboxybenzyl (Cbz), carbamate, 3,4-dimethoxy-benzyl (DMPM), 9-fluorenylmethyloxycarbonyl (Fmoc), p-methoxybenzyl carbonyl (Moz), 4-nitrobenzylsulfonyl (Nos), p-methoxybenzyl (PMB), p-methoxyphenyl (PMP), 4-toluenesulfonyl (Tos), and trichloroethyl chloroformate (Troc).

26. The process of claim 25, wherein the peptide coupling reagent is 1-[bis(dimethylamino)-methylene]-1H-1,2,3-triazolo[4,5-b]pyridinium 3-oxid hexafluorophosphate (HATU).

27. The process of claim 25 or 26, wherein the base is N,N-diisopropylethyl amine (DIPEA or Hunig's base).

28. The process of any one of claims 25-27, wherein the solvent is dimethylformamide.

29. The process of any one of claims 25-28, wherein R⁶ is tert-butyloxycarbonyl (Boc).

30. The process of any one of claims 25-29, wherein R⁴ is fluoro.

31. A process for preparing a compound of Formula VII: 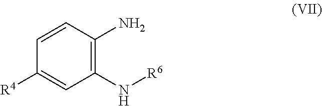
comprising treating a compound of Formula IX: 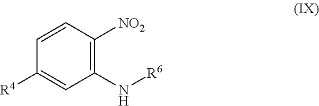
with hydrogen gas in the presence of a palladium catalyst and a solvent or mixture of solvents;
wherein
R⁴ is selected from the group consisting of fluoro, bromo, and chloro; and
R⁶ is a protecting group selected from the group consisting of acetyl (Ac), benzyl (Bn), tert-butyloxycarbonyl (Boc), benzoyl (Bz), carboxybenzyl (Cbz), carbamate, 3,4-dimethoxy-benzyl (DMPM), 9-fluorenylmethyloxycarbonyl (Fmoc), p-methoxybenzyl carbonyl (Moz), 4-nitrobenzylsulfonyl (Nos), p-methoxybenzyl (PMB), p-methoxyphenyl (PMP), 4-toluenesulfonyl (Tos), and trichloroethyl chloroformate (Troc).

32. The process of claim 31, wherein the palladium catalyst in 10% palladium on carbon.

33. The process of claim 31 or 32, wherein the solvent is a mixture of ethanol and ethyl acetate.

34. The process of any one of claims 31-33, wherein R⁴ is fluoro.

35. The process of any one of claims 31-34, wherein R⁶ is tert-butyloxycarbonyl (Boc).

36. A process for preparing a compound of Formula IX: 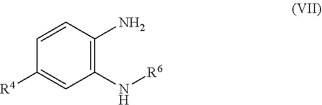
comprising reacting a compound of Formula X: 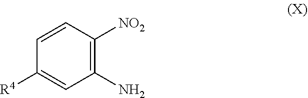
with a protecting group reagent and a base, or combination of bases, in a solvent:
wherein
R⁴ is selected from the group consisting of fluoro, bromo, and chloro; and
R⁶ is a protecting group selected from the group consisting of acetyl (Ac), benzyl (Bn), tert-butyloxycarbonyl (Boc), benzoyl (Bz), carboxybenzyl (Cbz), carbamate, 3,4-dimethoxy-benzyl (DMPM), 9-fluorenylmethyloxycarbonyl (Fmoc), p-methoxybenzyl carbonyl (Moz), 4-nitrobenzylsulfonyl (Nos), p-methoxybenzyl (PMB), p-methoxyphenyl (PMP), 4-toluenesulfonyl (Tos), and trichloroethyl chloroformate (Troc).

37. The process of claim 36, wherein the protecting group reagent is di-tert-butyl dicarbonate (Boc₂O) and R⁶ is tert-butyloxycarbonyl (Boc).

38. The process of claim 36 or 37, wherein the base is a combination of 4-dimethylaminopyridine (DMAP) and is N,N-diisopropylethyl amine (DIPEA or Hünig's base).

39. The process of any one of claims 36-38, wherein the solvent is dichloromethane (DCM).

40. The process of any one of claims 36-39, wherein R⁴ is fluoro.

41. A process for preparing a compound of Formula VIII: 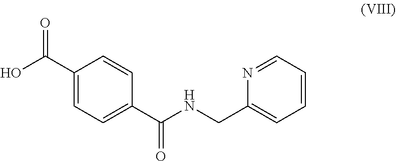 [SMILES: O=C(O)c1ccc(C(=O)NCc2ccccn2)cc1]
comprising reacting a compound of Formula XI: 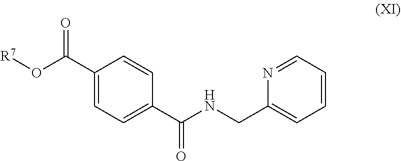
with a base in a solvent;
wherein R⁷ is C₁-C₆ alkyl.

42. The process of claim 41, wherein the base in sodium hydroxide (NaOH).

43. The process of claim 41 or 42, wherein the solvent is methanol.

44. The process of any one of claims 41-43, wherein R⁷ is —CH₃.

45. A process for preparing a compound of Formula XI: 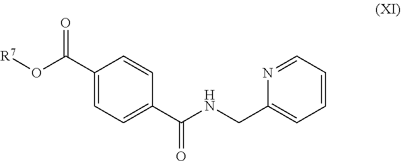
comprising reacting a compound of Formula XII: 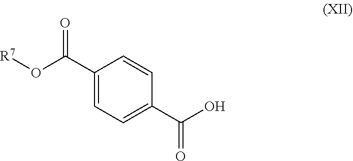
with a compound of Formula XIII: 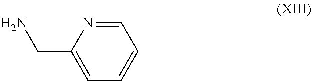
in the presence of a peptide coupling reagent, a base, and a solvent; wherein R⁷ is C₁-C₆ alkyl.

46. The process of claim 45, wherein the peptide coupling reagent is 1-[bis(dimethylamino)-methylene]-1H-1,2,3-triazolo[4,5-b]pyridinium 3-oxid hexafluorophosphate (HATU).

47. The process of claim 45 or 46, wherein the base is N,N-diisopropylethylamine (DIPEA or Hünig's base).

48. The process of any one of claims 45-47, wherein the solvent is dimethylformamide (DMF).

49. The process of any one of claims 45-48, wherein R⁷ is —CH₃.

50. A process for preparing a compound of Formula IV: 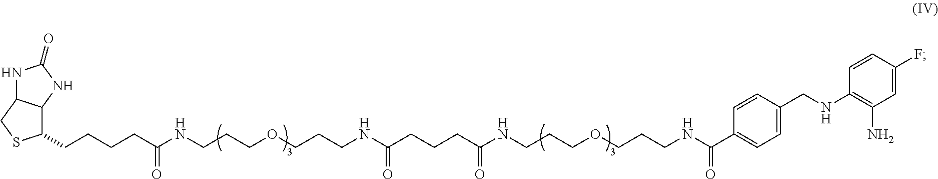
comprising reacting a compound of Formula XIV: 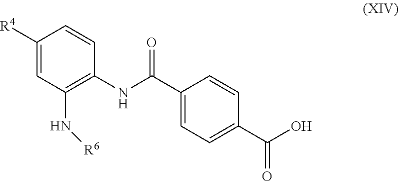
with a compound of Formula XV: 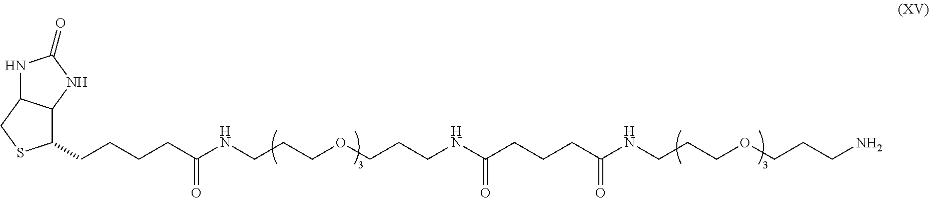
in the presence of a peptide coupling reagent and a base in a solvent, then further reacting the product of the above reaction with an acid;
wherein
R⁴ is selected from the group consisting of fluoro, bromo, and chloro; and
R⁶ is a protecting group selected from the group consisting of acetyl (Ac), benzyl (Bn), tert-butyloxycarbonyl (Boc), benzoyl (Bz), carboxybenzyl (Cbz), carbamate, 3,4-dimethoxy-benzyl (DMPM), 9-fluorenylmethyloxycarbonyl (Fmoc), p-methoxybenzyl carbonyl (Moz), 4-nitrobenzylsulfonyl (Nos), p-methoxybenzyl (PMB), p-methoxyphenyl (PMP), 4-toluenesulfonyl (Tos), and trichloroethyl chloroformate (Troc).

51. The process of claim 50, wherein the peptide coupling reagent is 1-[bis(dimethylamino)-methylene]-1H-1,2,3-triazolo[4,5-b]pyridinium 3-oxide hexafluorophosphate (HATU).

52. The process of claim 50 or 51, wherein the base is N,N-diisopropylethylamine (DIPEA or Hünig's base).

53. The process of any one of claims 50-52, wherein the solvent is dimethylformamide (DMF).

54. The process of any one of claims 50-53, wherein the acid is hydrochloric acid (HC).

55. The process of any one of claims 50-54, wherein R⁴ is fluoro and R⁶ is tert-butyloxycarbonyl (Boc).

56. A process for preparing a compound of Formula XIV: 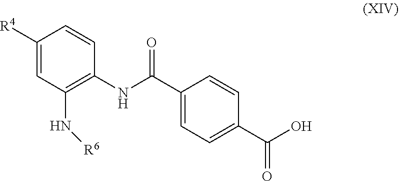
comprising reacting a compound of Formula XVI: 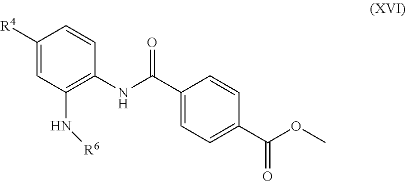
with a base in a solvent;
wherein
R⁴ is selected from the group consisting of fluoro, bromo, and chloro; and
R⁶ is a protecting group selected from the group consisting of acetyl (Ac), benzyl (Bn), tert-butyloxycarbonyl (Boc), benzoyl (Bz), carboxybenzyl (Cbz), carbamate, 3,4-dimethoxy-benzyl (DMPM), 9-fluorenylmethyloxycarbonyl (Fmoc), p-methoxybenzyl carbonyl (Moz), 4-nitrobenzylsulfonyl (Nos), p-methoxybenzyl (PMB), p-methoxyphenyl (PMP), 4-toluenesulfonyl (Tos), and trichloroethyl chloroformate (Troc).

57. The process of claim 56, wherein the base in sodium hydroxide (NaOH).

58. The process of claim 56 or 57, wherein the solvent is methanol (MeOH).

59. The process of any one of claims 56-58, wherein R⁴ is fluoro and R⁶ is tert-butyloxycarbonyl (Boc).

60. A process for preparing a compound of Formula XVI: 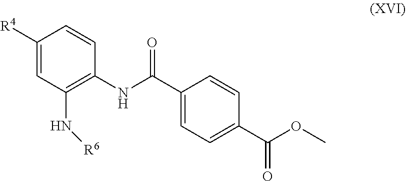
comprising reacting a compound of Formula XVII: 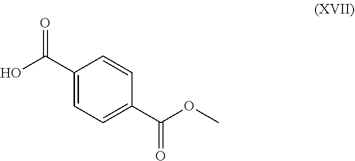
with a compound of Formula VII: 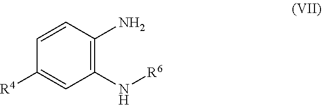
in the presence of a peptide coupling reagent and a base in a solvent;
wherein
R⁴ is selected from the group consisting of fluoro, bromo, and chloro; and
R⁶ is a protecting group selected from the group consisting of acetyl (Ac), benzyl (Bn), tert-butyloxycarbonyl (Boc), benzoyl (Bz), carboxybenzyl (Cbz), carbamate, 3,4-dimethoxy-benzyl (DMPM), 9-fluorenylmethyloxycarbonyl (Fmoc), p-methoxybenzyl carbonyl (Moz), 4-nitrobenzylsulfonyl (Nos), p-methoxybenzyl (PMB), p-methoxyphenyl (PMP), 4-toluenesulfonyl (Tos), and trichloroethyl chloroformate (Troc).

61. The process of claim 60, wherein the peptide coupling reagent is 1-[bis(dimethylamino)-methylene]-1H-1,2,3-triazolo[4,5-b]pyridinium 3-oxide hexafluorophosphate (HATU).

62. The process of claim 60 or 61, wherein the base is N,N-diisopropylethylamine (DIPEA or Hünig's base).

63. The process of any one of claims 60-62, wherein the solvent is dimethylformamide (DMF).

64. The process of any one of claims 60-63, wherein R⁴ is fluoro and R⁶ is tert-butyloxycarbonyl (Boc).

65. The compound of claim 1, wherein the compound of Formula I is selected from the group consisting of 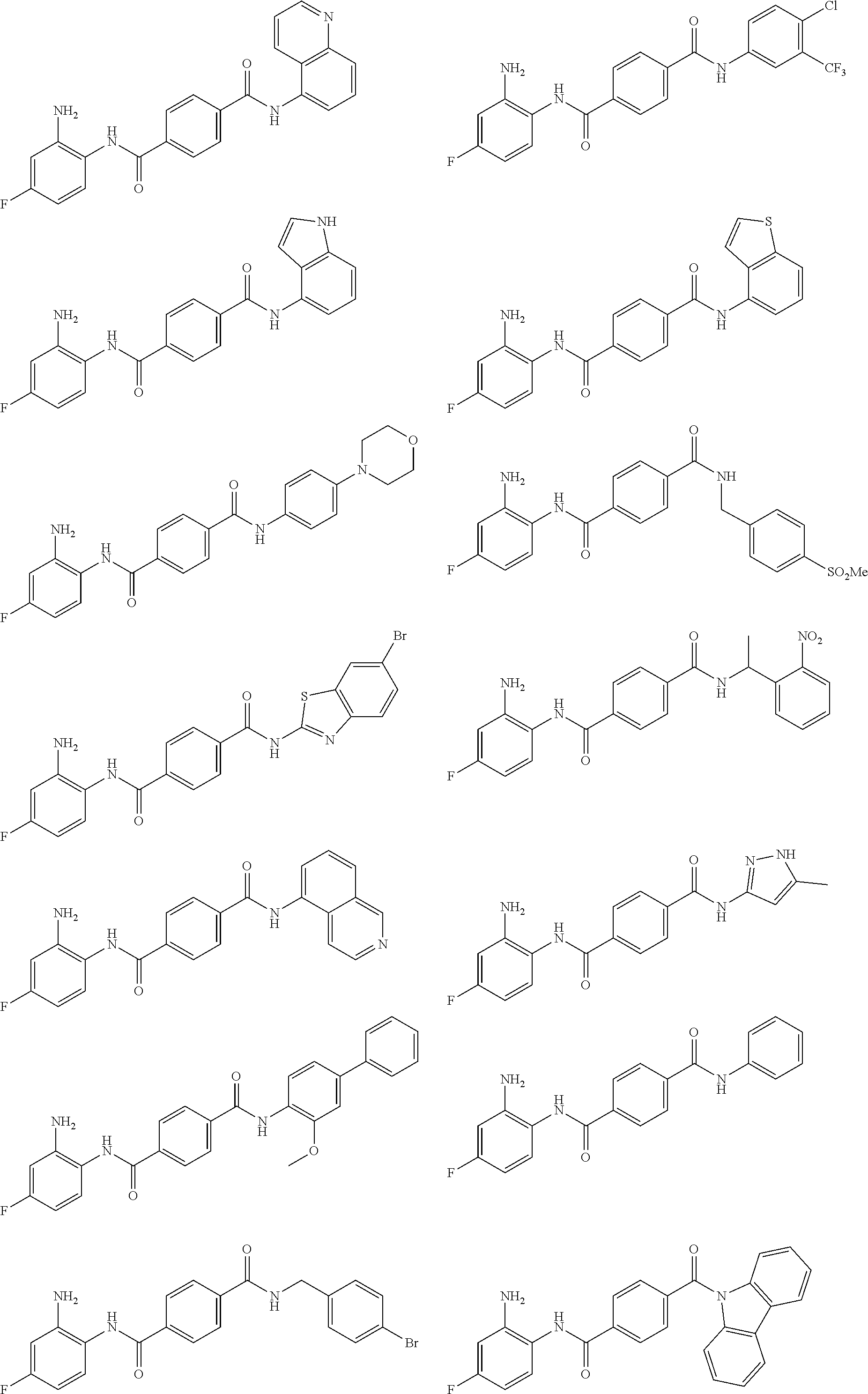 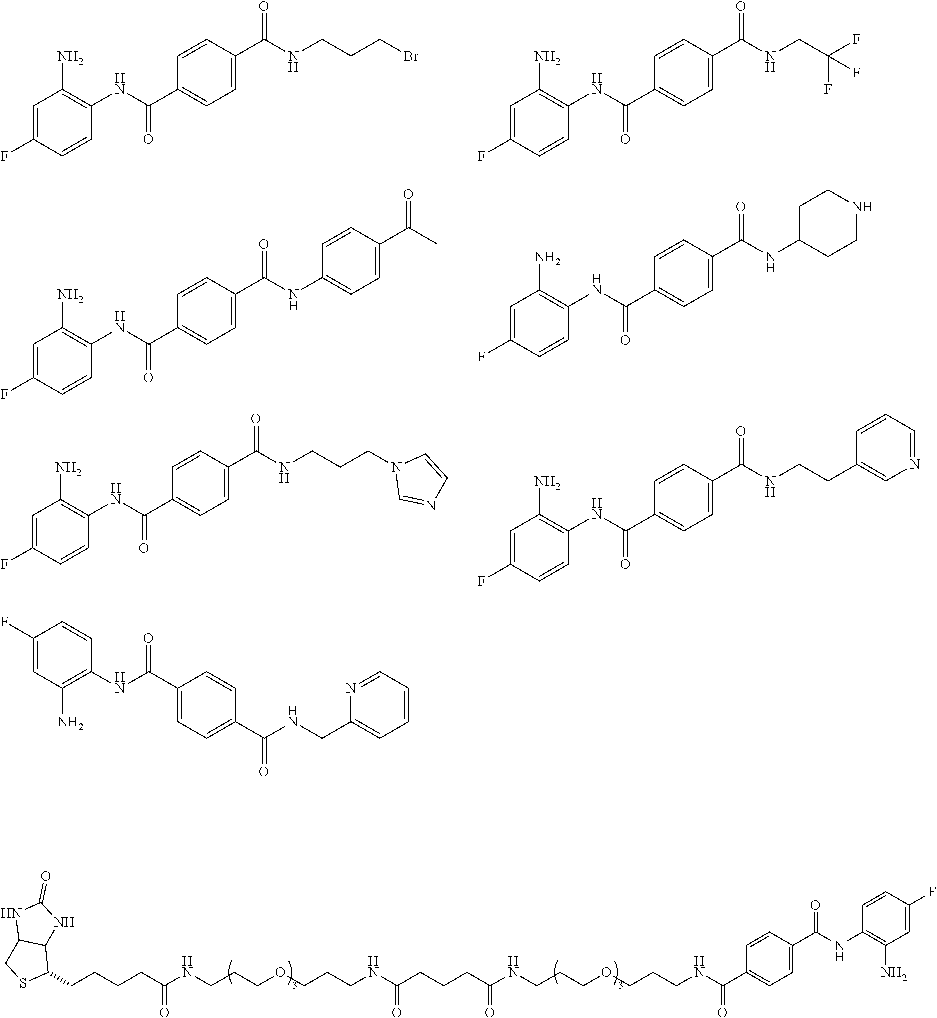
or a pharmaceutically acceptable sa thereof.
